(19) United States
(12) Patent Application Publication
(10) Pub. No.: US 2024/0226049 A1
(43) Pub. Date: Jul. 11, 2024

(54) METHODS OF TREATMENT USING BEMPEDOIC ACID

(71) Applicant: Esperion Therapeutics, Inc., Ann Arbor, MI (US)

(72) Inventors: Leanne Tyndall Bloedon, Raleigh, NC (US); William J. Sasiela, Bloomingdale, NJ (US)

(21) Appl. No.: 18/480,601

(22) Filed: Oct. 4, 2023

Related U.S. Application Data
(60) Provisional application No. 63/581,335, filed on Sep. 8, 2023.
(60) Provisional application No. 63/479,105, filed on Jan. 9, 2023.

(51) Int. Cl. A61K 31/20; A61K 31/397; A61P 9/10

(52) U.S. Cl. CPC A61K 31/20; A61K 31/397; A61P 9/10

(57) ABSTRACT

Provided herein are methods of reducing the risk of myocardial infarction or coronary revascularization in an adult with established cardiovascular disease or two or more risk factors for cardiovascular disease. Also provided herein are methods of reducing low-density lipoprotein cholesterol in an adult with primary hyperlipidemia. The methods described herein generally comprise administering to the adult an effective amount of bempedoic acid.

### CROSS-REFERENCE TO RELATED APPLICATIONS

This application claims priority to and the benefit of U.S. Provisional Patent Application No. 63/479,105, filed on Jan. 9, 2023 and U.S. Provisional Patent Application No. 63/581,335, filed on Sep. 8, 2023, the entire disclosures of each of which are hereby incorporated by reference herein in their entireties.

### BACKGROUND

Administration of statins for elevated levels of low-density lipoprotein-cholesterol (LDL-C) represents the cornerstone of contemporary therapy to reduce the risk of major adverse cardiovascular events in both primary and secondary prevention patients. However, many patients report adverse musculoskeletal side effects that either prevents them from using statins or limits their ability to tolerate a dosage necessary to achieve their cholesterol targets. Withdrawal from or failure to maximize statin therapy are associated with increased risk of adverse cardiovascular events. Consequently, there is an unmet need to develop novel therapeutic strategies for reducing LDL-C and minimizing the risk of major adverse cardiovascular events in patients with elevated levels of LDL-C.

### SUMMARY

The present disclosure provides methods of using bempedoic acid to reduce the risk of a major adverse cardiovascular event (MACE) (e.g., cardiovascular death, myocardial infarction, stroke, or coronary revascularization) in an adult at high risk for a cardiovascular disease event (e.g., an adult with established cardiovascular disease or two or more risk factors for cardiovascular disease). The disclosure additionally provides methods of using bempedoic acid to reduce low-density lipoprotein (LDL-C) in an adult with primary hyperlipidemia. The methods generally comprise administering to the adult a pharmaceutical formulation comprising an effective amount of bempedoic acid (e.g., 180 mg bempedoic acid).

In one aspect, the disclosure provides a method of reducing the risk of myocardial infarction in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof.

In another aspect, the disclosure provides a method of reducing the risk of coronary revascularization in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof.

In another aspect, the disclosure provides a method of reducing LDL-C in an adult with primary hyperlipidemia, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof, alone or in combination with another LDL-C-lowering therapy.

In another aspect, the disclosure provides a method of reducing LDL-C in an adult with primary hyperlipidemia, the method comprising orally administering daily to the adult a pharmaceutical formulation comprising 180 mg bempedoic acid; 10 mg ezetimibe; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof, alone or in combination with another LDL-C-lowering therapy.

In another aspect, the disclosure provides a method of reducing the risk of non-fatal myocardial infarction in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients, wherein administration of the pharmaceutical formulation reduces the risk of non-fatal myocardial infarction in the adult by about 27% as compared to an adult with established cardiovascular disease or without established cardiovascular disease and at high risk for a cardiovascular disease event administered a placebo.

In another aspect, the disclosure provides a method of reducing the risk of coronary revascularization in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients, wherein administration of the pharmaceutical formulation reduces the risk of coronary revascularization in the adult by about 19% as compared to an adult with established cardiovascular disease or without established cardiovascular disease and at high risk for a cardiovascular disease event administered a placebo.

In another aspect, the disclosure provides a method of treating an adult at risk for a MACE, the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 15% as compared to an adult at risk for the MACE administered a placebo, and wherein the MACE is cardiovascular death, non-fatal myocardial infarction, or non-fatal stroke (i.e., MACE-3).

In another aspect, the disclosure provides a method of treating an adult at risk for a MACE, the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 13% as compared to an adult at risk for the MACE administered a placebo, and wherein the MACE is cardiovascular death, non-fatal myocardial infarction, non-fatal stroke, or coronary revascularization (i.e., MACE-4).

It should be understood where administration of the pharmaceutical formulation of the present teachings is compared to an adult administered a placebo that the administration of the pharmaceutical formulation can be compared to a placebo in a human population, e.g., compared to a placebo in a human population with established cardiovascular disease or without established cardiovascular disease and at high risk for a cardiovascular disease event. In certain embodiments, administration of the pharmaceutical formulation of the present teachings reduces the risk of an adverse event as described herein as compared to a placebo in a human population with established cardiovascular disease or without established cardiovascular disease and at high risk for a cardiovascular disease event.

In another aspect, the disclosure provides a method of reducing the risk of a MACE in an adult at high risk for a cardiovascular disease event, the method comprising orally administering daily to the adult a pharmaceutical formulation comprising 180 mg bempedoic acid and one or more pharmaceutically acceptable excipients, wherein the adult has no prior cardiovascular events.

### BRIEF DESCRIPTION OF FIGURES

FIG. 1A shows the percent change from baseline in low-density lipoprotein cholesterol (LDL-C) for the bempedoic acid and placebo treatment groups at time points during the study, as further described in Example 1. The mean starting LDL-C for both groups was 139.0 mg/dL. To convert the values for cholesterol to millimoles per liter, multiply by 0.02586.

FIG. 1B shows the changes in high-sensitivity C-reactive protein (hsCRP) for the bempedoic acid and placebo treatment groups at timepoints during the study. The median starting hsCRP was 2.3 mg/L.

FIG. 2A shows the cumulative incidence of the primary efficacy end point (a composite of death from coronary causes, nonfatal myocardial infarction, nonfatal stroke, or coronary revascularization) for bempedoic acid and placebo treatment groups, as further described in Example 1. The insets show the same data on an enlarged y axis. The P values were calculated with the use of log-rank tests. MACE denotes major adverse cardiovascular events.

FIG. 2B shows the cumulative incidence of the key secondary endpoint (a composite of death from cardiovascular causes, nonfatal myocardial infarction, or nonfatal stroke) for bempedoic acid and placebo treatment groups, as further described in Example 1. The insets show the same data on an enlarged y axis. The P values were calculated with the use of log-rank tests. MACE denotes major adverse cardiovascular events.

FIG. 2C shows the cumulative incidence of the key secondary end point (fatal and nonfatal myocardial infarction (MI)) for bempedoic acid and placebo treatment groups, as further described in Example 1. The insets show the same data on an enlarged y axis. The P values were calculated with the use of log-rank tests.

FIG. 2D shows the cumulative incidence of the key secondary endpoint (coronary revascularization) for bempedoic acid and placebo treatment groups, as further described in Example 1. The insets show the same data on an enlarged y axis. The P values were calculated with the use of log-rank tests.

FIG. 3 describes the flow of patients through the study described in Example 2.

FIG. 4 shows a table describing the effect of trial regimens (e.g., bempedoic acid and placebo) on lipid and inflammatory biomarkers, as further described in Example 2.

FIG. 5 shows the percent change from baseline in low-density lipoprotein cholesterol (LDL-C) for the bempedoic acid and placebo treatment groups at time points during the study, as further described in Example 2.

FIG. 6A shows the time to incidence of a primary end-point event, a four-component composite of major adverse cardiovascular events (MACE) that included death from cardiovascular causes, nonfatal myocardial infarction, nonfatal stroke, or coronary revascularization for bempedoic acid and placebo treatment groups, as further described in Example 2. The P values were calculated with the use of the log-rank test. The median follow up time was 39.9 months.

FIG. 6B shows the time to first incidence of a three-component MACE that included death from cardiovascular causes, nonfatal myocardial infarction, or nonfatal stroke for bempedoic acid and placebo treatment groups, as further described in Example 2. The P values were calculated with the use of the log-rank test. The median follow up time was 39.9 months.

FIG. 6C shows the time to first incidence of fatal or nonfatal myocardial infarction for bempedoic acid and placebo treatment groups, as further described in Example 2. The P values were calculated with the use of the log-rank test. The median follow up time was 39.9 months.

FIG. 6D shows the time to first incidence of fatal or nonfatal stroke for bempedoic acid and placebo treatment groups, as further described in Example 2. The P values were calculated with the use of the log-rank test. The median follow up time was 39.9 months.

FIG. 6E shows the time to first incidence of cardiovascular death for bempedoic acid and placebo treatment groups, as further described in Example 2. The P values were calculated with the use of the log-rank test. The median follow up time was 39.9 months.

FIG. 6F shows the time to first incidence of all-cause mortality for bempedoic acid and placebo treatment groups, as further described in Example 2. The P values were calculated with the use of the log-rank test. The median follow up time was 39.9 months.

FIG. 7 shows the time to first occurrence of any component of five-component MACE for bempedoic acid and placebo treatment groups, as further described in Example 2.

FIG. 8 shows the time to first occurrence of coronary revascularization for bempedoic acid and placebo treatment groups, as further described in Example 2.

FIG. 9 shows the time to first occurrence of hospitalization for unstable angina for bempedoic acid and placebo treatment groups, as further described in Example 2.

### DETAILED DESCRIPTION

As generally described herein, the disclosure provides methods of reducing the risk of a major adverse cardiovascular event (MACE) (e.g., cardiovascular death, myocardial infarction, stroke, or coronary revascularization) in an adult at high risk for a cardiovascular disease event (e.g., an adult with established cardiovascular disease or two or more risk factors for cardiovascular disease). The disclosure additionally provides methods of reducing low-density lipoprotein (LDL-C) in an adult with primary hyperlipidemia. The methods generally comprise administering to the adult a pharmaceutical formulation comprising an effective amount of bempedoic acid (e.g., 180 mg bempedoic acid).

### Definitions

To facilitate an understanding of the present disclosure, a number of terms and phrases are defined below.

Unless defined otherwise, all technical and scientific terms used herein have the same meaning as commonly understood by one of ordinary skill in the art to which this disclosure belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” refers to one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10%, ±5%, ±2%, or ±1% variation from the nominal value unless otherwise indicated or inferred from the context.

It should be understood that the order of steps or order for performing certain actions is immaterial so long as the present disclosure remains operable. Moreover, two or more steps or actions may be conducted simultaneously.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a carrier, inert or active, making the composition especially suitable for diagnostic or therapeutic use in vivo or ex vivo.

The phrases “pharmaceutically acceptable” and “pharmacologically acceptable,” as used herein, refer to compounds, molecular entities, compositions, materials, and/or dosage forms that do not produce an adverse, allergic, or other untoward reaction when administered to an animal, or a human, as appropriate. For human administration, preparations should meet sterility, pyrogenicity, and general safety and purity standards as required by regulatory agencies that evaluate the safety and efficacy of pharmaceuticals and drug products, e.g., the U.S. Food and Drug Administration. “Pharmaceutically acceptable” and “pharmacologically acceptable” can mean approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present disclosure (e.g., bempedoic acid), which salt is compatible with pharmaceutical administration. For example, one or both of the carboxylic acid groups of bempedoic acid can be transformed to pharmaceutically acceptable salt(s).

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “carrier” refers to a material, composition, or vehicle, such as a liquid or solid filler, diluent, excipient, solvent, or encapsulating material, involved in carrying or transporting a pharmaceutical agent such as bempedoic acid, or a pharmaceutically acceptable salt thereof, from one organ, or portion of the body, to another organ, or portion of the body.

As used herein, “pharmaceutically acceptable excipient” refers to a substance that aids the administration of an active agent to and/or absorption by a subject (e.g., an adult) and can be included in the compositions of the present disclosure without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. For examples of excipients, see Martin, Remington's Pharmaceutical Sciences, 15th Ed., Mack Publ. Co., Easton, PA (1975).

As used herein, “treat,” “treating” or “treatment” includes an action that occurs while a subject (e.g., an adult) is suffering from a specified disease, disorder or condition, which reduces the severity of the disease, disorder or condition, or retards or slows the progression of the disease, disorder or condition (e.g., lessening, reducing, modulating, ameliorating or eliminating, that results in the improvement of the condition, disease, disorder, and the like), or lessens, reduces, modulates, ameliorates or eliminates a symptom thereof. Treating can be curing, improving, or at least partially ameliorating the disorder. In certain embodiments, treating is curing the disease.

As used herein, “reducing” or “reduction” of a symptom or symptoms (and grammatical equivalents of this phrase) refers to decreasing of the severity or frequency of the symptom(s), or elimination of the symptom(s).

As used herein, “reducing” or “reduction” of an elevated laboratory biomarker/parameter or vital sign associated with a disease disclosed herein may refer a decrease in the elevated laboratory biomarker/parameter or vital sign, for example, to a predetermined clinically relevant endpoint (e.g., a clinically normal level).

As used herein, “subject” and “patient” are used interchangeably and refer to an organism to be treated by the methods and compositions of the present disclosure. Such organisms are preferably a mammal (e.g., human, mouse, rat, guinea pig, dog, cat, horse, cow, pig, or non-human primate, such as a monkey, chimpanzee, baboon, and rhesus), and more preferably, a human. In certain embodiments, the subject is an adult human.

As used herein, “solid dosage form” refers to a pharmaceutical dose(s) in solid form, e.g., tablets, capsules, granules, powders, sachets, reconstitutable powders, dry powder inhalers, and chewables.

As used herein, “immediate release” refers to a dosage form that has not been engineered to modify or control the release of the active ingredient.

As used herein, “sustained release” refers to a dosage form designed to release a drug at a predetermined (but not necessarily constant) rate in order to maintain a desired range of drug concentration over a specific period of time, e.g., 8 hours, 12 hours, 16 hours, 20 hours, 24 hours, etc., with minimum side effects.

As used herein, “fixed-dose combination” refers to a form in which the active ingredients (e.g., bempedoic acid and ezetimibe) are both administered to a patient simultaneously in the form of a single entity or dosage.

As used herein, “administering” refers to oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject (e.g., an adult). Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., bempedoic acid and ezetimibe). Bempedoic acid, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, “disease,” “disorder,” “condition,” or “illness,” which can be used interchangeably herein unless otherwise understood from the context, refers to a state of being or health status of a patient or subject (e.g., an adult) capable of being treated with a compound, pharmaceutical material, pharmaceutical composition, or method provided herein.

As used herein, “effective amount” or “therapeutically-effective amount” refers to the amount of a compound (e.g., bempedoic acid), a combination of compounds (e.g., bempedoic acid and ezetimibe), a pharmaceutical composition (e.g., a pharmaceutical composition of the present disclosure), or a fixed-dose combination (e.g., a fixed-dose combination of the present disclosure) sufficient to effect beneficial or desired results. An effective amount can be administered in one or more administrations, applications or dosages and is not intended to be limited to a particular formulation or administration route.

### Bempedoic Acid

Bempedoic acid is a non-statin drug indicated as an adjunct to diet and maximally tolerated statin therapy for the treatment of adults with heterozygous familial hypercholesterolemia or established atherosclerotic cardiovascular disease who require additional lowering of LDL-C. It functions through inhibition of adenosine triphosphate-citrate lyase (ACL). It behaves as a prodrug in vivo, where it is converted to the active species bempedoic acid-CoA by endogenous liver acyl-Coenzyme (CoA) synthetase (ACS) activity. A specific ACS isozyme, very long-chain acyl-CoA synthetase (ACSVL1), is required to form the active species. Bempedoic acid can also activate the metabolic sensor AMP-activated protein kinase (AMPK).

Bempedoic acid may also be represented by the structure of Formula (I):

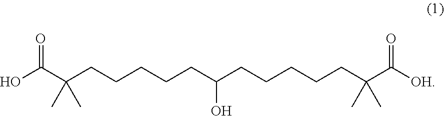 [SMILES: CC(C)(CCCCCC(O)CCCCCC(C)(C)C(=O)O)C(=O)O]

Bempedoic acid and a process for synthesizing it are disclosed in the U.S. Pat. Nos. 7,335,799 and 11,407,705; and International Publication No. WO 2020/257571 A1, each of which is herein incorporated by reference. Bempedoic acid may also be referred to as ETC-1002, ESP-55016, or under the tradenames Nexletol® and Nilemdo®.

In various embodiments, bempedoic acid may be used for the treatment and/or prevention of a variety of conditions, diseases and disorders described herein. The methods of treating a condition, disease, or disorder described herein generally comprise administering to an adult in need thereof, a therapeutically effective amount of bempedoic acid to treat the condition, disease, or disorder.

In certain embodiments, bempedoic acid may be used for reducing the risk of myocardial infarction in an adult with established cardiovascular disease or two or more risk factors for cardiovascular disease.

In certain embodiments, bempedoic acid may be used for reducing the risk of non-fatal myocardial infarction in an adult with established cardiovascular disease or without established cardiovascular disease and at high risk for a cardiovascular disease event.

In certain embodiments, bempedoic acid may be used for reducing the risk of coronary revascularization in an adult with established cardiovascular disease or two or more risk factors for cardiovascular disease.

In certain embodiments, bempedoic acid may be used for reducing the risk of coronary revascularization in an adult with established cardiovascular disease or without established cardiovascular disease and at high risk for a cardiovascular disease event.

In certain embodiments, bempedoic acid may be used for reducing low-density lipoprotein cholesterol (LDL-C) in an adult with primary hyperlipidemia.

In certain embodiments, bempedoic acid may be used for treating an adult at risk for a major adverse cardiovascular event (MACE). In certain embodiments, the MACE is cardiovascular death, non-fatal myocardial infarction, or non-fatal stroke (i.e., MACE-3). In certain embodiments, the MACE is cardiovascular death, non-fatal myocardial infarction, non-fatal stroke, or coronary revascularization (i.e., MACE-4).

In certain embodiments, bempedoic acid may be used for reducing the risk of a MACE in an adult at high risk for a cardiovascular disease event. In certain embodiments, the adult has no prior cardiovascular events.

In certain embodiments, the fixed-dose combinations and pharmaceutical formulations disclosed herein comprise a crystalline form of bempedoic acid. As used herein, “crystalline form of bempedoic acid” may refer to a crystalline form of the free acid form of bempedoic acid or a crystalline form of a pharmaceutically acceptable salt of bempedoic acid. See, e.g., International Publication No. WO 2020/257573 A1, which is herein incorporated by reference.

In certain embodiments, the fixed-dose combinations and pharmaceutical formulations disclosed herein comprise a high purity crystalline form of bempedoic acid.

In certain embodiments, the fixed-dose combinations and pharmaceutical formulations disclosed herein comprise a pharmaceutical material comprising bempedoic acid.

In various embodiments, a pharmaceutical material generally comprises a crystalline form of bempedoic acid, wherein the pharmaceutical material comprises bempedoic acid, or a pharmaceutically acceptable salt thereof, in an amount greater than 99.0% by weight based on the total weight of the pharmaceutical material. In some embodiments, the amount of bempedoic acid, or a pharmaceutically acceptable salt thereof, in the pharmaceutical material is greater than about 99.1%, greater than about 99.2%, greater than about 99.3%, greater than about 99.4%, greater than about 99.5%, greater than about 99.6%, greater than about 99.7%, greater than about 99.8%, greater than about 99.85%, greater than about 99.9%, greater than about 99.95%, or greater than about 99.98% by weight of the total weight of the pharmaceutical material. In some embodiments, the pharmaceutical material comprises bempedoic acid, or a pharmaceutically acceptable salt thereof, in an amount greater than 99.5% by weight based on the total weight of the pharmaceutical material. In some embodiments, the pharmaceutical material comprises bempedoic acid, or a pharmaceutically acceptable salt thereof, in an amount greater than 99.7% by weight based on the total weight of the pharmaceutical material. In some embodiments, the pharmaceutical material comprises bempedoic acid, or a pharmaceutically acceptable salt thereof, in an amount greater than 99.9% by weight based on the total weight of the pharmaceutical material.

In various embodiments, a pharmaceutical material generally comprises a crystalline form of bempedoic acid, wherein the pharmaceutical material comprises bempedoic acid in an amount greater than 99.0% by weight based on the total weight of the pharmaceutical material. In some embodiments, the amount of bempedoic acid in the pharmaceutical material is greater than about 99.1%, greater than about 99.2%, greater than about 99.3%, greater than about 99.4%, greater than about 99.5%, greater than about 99.6%, greater than about 99.7%, greater than about 99.8%, greater than about 99.85%, greater than about 99.9%, greater than about 99.95%, or greater than about 99.98% by weight of the total weight of the pharmaceutical material. In some embodiments, the pharmaceutical material comprises bempedoic acid in an amount greater than 99.5% by weight based on the total weight of the pharmaceutical material. In some embodiments, the pharmaceutical material comprises bempedoic acid in an amount greater than 99.7% by weight based on the total weight of the pharmaceutical material. In some embodiments, the pharmaceutical material comprises bempedoic acid in an amount greater than 99.9% by weight based on the total weight of the pharmaceutical material.

In certain embodiments, the pharmaceutical material comprises bempedoic acid in an amount of from about 98% to about 102% by weight based on the total weight of the pharmaceutical material (anhydrous, solvent-free basis), as determined by a high-performance liquid chromatography (HPLC) assay.

In certain embodiments, the HPLC assay comprises one or more of:
(i) a Waters XBridge BEH C18 column (4.6 mm i.d.×150 mm, 2.5 pm);
(ii) a column temperature of about 40° C.;
(iii) a mobile phase comprising about 0.05% phosphoric acid in water/acetonitrile (about 50:50);
(iv) isocratic elution;
(v) a flow rate of about 1.2 mL/minute;
(vi) a sample temperature of ambient temperature;
(vii) detection at 215 nm; and
(viii) the retention time of the compound of formula (I) is about 4.6 minutes. In some embodiments, the HPLC assay comprises each of the above, i.e., (i)-(viii).

In certain embodiments, the crystalline form of bempedoic acid may be a crystalline form of bempedoic acid as characterized in International Publication Nos.

WO 2020/257571 A1 and WO 2020/257573 A1, each of which is herein incorporated by reference. A crystalline form of bempedoic acid may be characterized, for example, by an X-ray powder diffraction pattern or peak(s), and/or other characteristic properties such as melting point and hygroscopicity. Crystalline forms of bempedoic acid may include, but are not limited to, cocrystals (e.g., an aspartame cocrystal and a palmitic acid cocrystal), crystalline salts (e.g., an ammonium salt, a sodium salt, a potassium salt, a calcium salt, a lysine salt, a diethylamine salt, an ethylenediamine salt, a piperazine salt, a betaine salt, a tromethamine salt, and an isonicotinamide salt).

### Ezetimibe

Ezetimibe is a cholesterol absorption inhibitor indicated to reduce total cholesterol, low-density lipoprotein (LDL), apolipoprotein B (apo B), and non-high-density lipoprotein (HDL) in patients with primary hyperlipidemia, mixed hyperlipidemia, familial hypercholesterolemia (FH), and homozygous sitosterolemia (phytosterolemia).

Ezetimibe may be used in the fixed-dose combinations, pharmaceutical compositions, and methods of treatment described herein. In various embodiments, the fixed-dose combinations and pharmaceutical compositions provided herein comprise ezetimibe. Ezetimibe is represented by the structure of Formula (II):

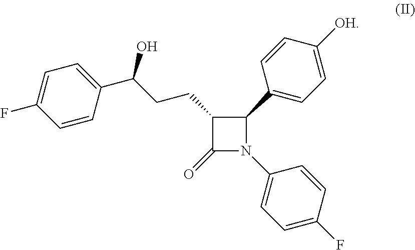 [SMILES: O=C1[C@H](CC[C@H](O)c2ccc(F)cc2)[C@@H](c2ccc(O)cc2)N1c1ccc(F)cc1]

Ezetimibe and its process of manufacture are disclosed in, for example, U.S. Pat. Nos. 5,631,365, which is incorporated herein by reference. Ezetimibe may be administered as an oral dosage form (e.g., a tablet). Ezetimibe may also be referred to as (3R,4S)-1-(4-fluorophenyl)-3-[(3S)-3-(4-fluorophenyl)-3-hydroxypropyl]-4-(4-hydroxyphenyl)azetidin-2-one, or under the tradenames Zetia® and Ezetrol®.

In various embodiments, ezetimibe may be used for the treatment or prevention of a variety of conditions, diseases, and disorders described herein. The methods of preventing or treating a condition, disease, or disorder described herein generally comprise administering to an adult a therapeutically effective amount of ezetimibe to prevent or treat the condition, disease, or disorder.

In certain embodiments, ezetimibe may be used in combination with bempedoic acid for reducing low-density lipoprotein cholesterol (LDL-C) in an adult with primary hyperlipidemia.

### Fixed-Dose Combinations

Disclosed herein are fixed-dose combinations generally comprising bempedoic acid and ezetimibe. A fixed-dose combination of bempedoic acid and ezetimibe may also be referred to herein under the tradenames Nexlizet® and Nustendi®.

In various embodiments, a fixed-dose combination described herein is a solid dosage form comprising bempedoic acid and ezetimibe.

In various embodiments, a fixed-dose combination described herein is a solid dosage form comprising a pharmaceutical formulation described herein.

In certain embodiments, the fixed-dose combination comprises about 180 mg bempedoic acid. In some embodiments, the fixed-dose combination comprises 180 mg bempedoic acid.

In some embodiments, the fixed-dose combination comprises about 10 mg ezetimibe. In some embodiments, the fixed-dose combination comprises 10 mg ezetimibe.

In certain embodiments, the fixed-dose combination comprises about 180 mg bempedoic acid and about 10 mg ezetimibe. In some embodiments, the fixed-dose combination comprises 180 mg bempedoic acid and 10 mg ezetimibe.

In some embodiments, the fixed-dose combinations disclosed herein are formulated for oral delivery. In some embodiments, the fixed-dose combinations disclosed herein are formulated as an oral dosage form. Examples of oral dosage forms include, but are not limited to, a drench, a tablet, a capsule, a softgel capsule, a cachet, a pill, an emulsion, a lozenge, a solution, a suspension, a bolus, a powder, an elixir or syrup, a pastille, a mouthwash, a granule, or a paste for application to the tongue. In some embodiments, the fixed-dose combination is formulated as a tablet.

In certain embodiments, the fixed-dose combinations provided herein can be used for the treatment of a disease, disorder, or condition described herein.

In certain embodiments, a fixed-dose combination described herein may be used for reducing the risk of myocardial infarction in an adult with established cardiovascular disease or two or more risk factors for cardiovascular disease.

In certain embodiments, a fixed-dose combination described herein may be used for reducing the risk of non-fatal myocardial infarction in an adult with established cardiovascular disease or without established cardiovascular disease and at high risk for a cardiovascular disease event.

In certain embodiments, a fixed-dose combination described herein may be used for reducing the risk of coronary revascularization in an adult with established cardiovascular disease or two or more risk factors for cardiovascular disease.

In certain embodiments, a fixed-dose combination described herein may be used for reducing the risk of coronary revascularization in an adult with established cardiovascular disease or without established cardiovascular disease and at high risk for a cardiovascular disease event.

In certain embodiments, a fixed-dose combination described herein may be used for reducing low-density lipoprotein cholesterol (LDL-C) in an adult with primary hyperlipidemia.

In certain embodiments, a fixed-dose combination described herein may be used for treating an adult at risk for a major adverse cardiovascular event (MACE). In certain embodiments, the MACE is cardiovascular death, non-fatal myocardial infarction, or non-fatal stroke (i.e., MACE-3). In certain embodiments, the MACE is cardiovascular death, non-fatal myocardial infarction, non-fatal stroke, or coronary revascularization.

In certain embodiments, a fixed-dose combination described herein may be used for reducing the risk of a major adverse cardiovascular event (MACE) in an adult at high risk for a cardiovascular disease event, where the adult has no prior cardiovascular events.

### Pharmaceutical Formulations

Also disclosed herein are pharmaceutical formulations generally comprising bempedoic acid, and one or more pharmaceutically acceptable excipients.

In certain embodiments, the one or more pharmaceutically acceptable excipients is selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof.

In various embodiments, provided herein is a pharmaceutical formulation comprising bempedoic acid; colloidal silicon dioxide; hydroxyl propyl cellulose; lactose monohydrate; magnesium stearate; microcrystalline cellulose; and sodium starch glycolate.

In various embodiments, provided herein is a pharmaceutical formulation comprising bempedoic acid; colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the pharmaceutical formulation further comprises ezetimibe.

In various embodiments, provided herein is a pharmaceutical formulation comprising bempedoic acid; ezetimibe; colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the amount of bempedoic acid in a pharmaceutical formulation described herein is about 45% (w/w) to about 55% (w/w). In certain embodiments, the amount of bempedoic acid in a pharmaceutical formulation described herein is about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), or about 55% (w/w). In certain embodiments, the amount of bempedoic acid in a pharmaceutical formulation described herein is about 47% (w/w). In certain embodiments, the amount of bempedoic acid in a pharmaceutical formulation described herein is about 48% (w/w). In certain embodiments, the amount of bempedoic acid in a pharmaceutical formulation described herein is about 49% (w/w). In certain embodiments, the amount of bempedoic acid in a pharmaceutical formulation described herein is about 50% (w/w). In certain embodiments, the amount of bempedoic acid in a pharmaceutical formulation described herein is about 51% (w/w). In certain embodiments, the amount of bempedoic acid in a pharmaceutical formulation described herein is about 52% (w/w).

In certain embodiments, the amount of bempedoic acid in a pharmaceutical formulation described herein is 47% (w/w), 47.1% (w/w), 47.2% (w/w), 47.3% (w/w), 47.4% (w/w), 47.5% (w/w), 47.6% (w/w), 47.7% (w/w), 47.8% (w/w), 47.9% (w/w), 48% (w/w), 48.1% (w/w), 48.2% (w/w), 48.3% (w/w), 48.4% (w/w), 48.5% (w/w), 48.6% (w/w), 48.7% (w/w), 48.8% (w/w), 48.9% (w/w), 49% (w/w), 49.1% (w/w), 49.2% (w/w), 49.3% (w/w), 49.4% (w/w), 49.5% (w/w), 49.6% (w/w), 49.7% (w/w), 49.8% (w/w), 49.9% (w/w), 50% (w/w), 50.1% (w/w), 50.2% (w/w), 50.3% (w/w), 50.4% (w/w), 50.5% (w/w), 50.6% (w/w), 50.7% (w/w), 50.8% (w/w), 50.9% (w/w), 51% (w/w), 51.1% (w/w), 51.2% (w/w), 51.3% (w/w), 51.4% (w/w), 51.5% (w/w), 51.6% (w/w), 51.7% (w/w), 51.8% (w/w), 51.9% (w/w), or 52% (w/w).

In certain embodiments, the amount of bempedoic acid in a pharmaceutical formulation described herein is about 180 mg. In certain embodiments, the amount of bempedoic acid in a pharmaceutical formulation described herein is 180 mg.

In certain embodiments, the amount of ezetimibe in a pharmaceutical formulation described herein is 0% (w/w) to about 3% (w/w). In certain embodiments, the amount of ezetimibe in a pharmaceutical formulation described herein is about 2.5% (w/w). In certain embodiments, the amount of ezetimibe in a pharmaceutical formulation described herein is about 2.6% (w/w). In certain embodiments, the amount of ezetimibe in a pharmaceutical formulation described herein is about 2.7% (w/w). In certain embodiments, the amount of ezetimibe in a pharmaceutical formulation described herein is about 2.8% (w/w). In certain embodiments, the amount of ezetimibe in a pharmaceutical formulation described herein is about 2.9% (w/w). In certain embodiments, the amount of ezetimibe in a pharmaceutical formulation described herein is about 3.0% (w/w).

In certain embodiments, the amount of ezetimibe in a pharmaceutical formulation described herein is about 10 mg. In certain embodiments, the amount of ezetimibe in a pharmaceutical formulation described herein is 10 mg.

In certain embodiments, the amount of colloidal silicon dioxide in a pharmaceutical formulation described herein is about 1% (w/w) to about 2% (w/w). In certain embodiments, the amount of colloidal silicon dioxide in a pharmaceutical formulation described herein is about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), or about 2% (w/w). In certain embodiments, the amount of colloidal silicon dioxide in a pharmaceutical formulation described herein is about 1% (w/w). In certain embodiments, the amount of colloidal silicon dioxide in a pharmaceutical formulation described herein is about 1.1% (w/w). In certain embodiments, the amount of colloidal silicon dioxide in a pharmaceutical formulation described herein is about 1.2% (w/w). In certain embodiments, the amount of colloidal silicon dioxide in a pharmaceutical formulation described herein is about 1.3% (w/w). In certain embodiments, the amount of colloidal silicon dioxide in a pharmaceutical formulation described herein is about 1.4% (w/w). In certain embodiments, the amount of colloidal silicon dioxide in a pharmaceutical formulation described herein is about 1.5% (w/w).

In certain embodiments, the amount of colloidal silicon dioxide in a pharmaceutical formulation described herein is about 3 mg to about 6 mg. In certain embodiments, the amount of colloidal silicon dioxide in a pharmaceutical formulation described herein is about 3 mg, about 3.5 mg, about 4 mg, about 4.5 mg, about 5 mg, about 5.5 mg, or about 6 mg. In certain embodiments, the amount of colloidal silicon dioxide in a pharmaceutical formulation described herein is about 3 mg. In certain embodiments, the amount of colloidal silicon dioxide in a pharmaceutical formulation described herein is about 3.5 mg. In certain embodiments, the amount of colloidal silicon dioxide in a pharmaceutical formulation described herein is about 4 mg. In certain embodiments, the amount of colloidal silicon dioxide in a pharmaceutical formulation described herein is about 4.5 mg. In certain embodiments, the amount of colloidal silicon dioxide in a pharmaceutical formulation described herein is about 5 mg. In certain embodiments, the amount of colloidal silicon dioxide in a pharmaceutical formulation described herein is about 5.5 mg. In certain embodiments, the amount of colloidal silicon dioxide in a pharmaceutical formulation described herein is about 6 mg.

In certain embodiments, the amount of the hydroxyl propyl cellulose in a pharmaceutical formulation described herein is about 3% (w/w) to about 5% (w/w). In certain embodiments, the amount of the hydroxyl propyl cellulose in a pharmaceutical formulation described herein is about 3% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), or about 5% (w/w). In certain embodiments, the amount of the hydroxyl propyl cellulose in a pharmaceutical formulation described herein is about 3% (w/w). In certain embodiments, the amount of the hydroxyl propyl cellulose in a pharmaceutical formulation described herein is about 3.1% (w/w). In certain embodiments, the amount of the hydroxyl propyl cellulose in a pharmaceutical formulation described herein is about 3.2% (w/w). In certain embodiments, the amount of the hydroxyl propyl cellulose in a pharmaceutical formulation described herein is about 3.3% (w/w). In certain embodiments, the amount of the hydroxyl propyl cellulose in a pharmaceutical formulation described herein is about 3.4% (w/w). In certain embodiments, the amount of the hydroxyl propyl cellulose in a pharmaceutical formulation described herein is about 3.5% (w/w). In certain embodiments, the amount of the hydroxyl propyl cellulose in a pharmaceutical formulation described herein is about 4.6% (w/w). In certain embodiments, the amount of the hydroxyl propyl cellulose in a pharmaceutical formulation described herein is about 4.7% (w/w). In certain embodiments, the amount of the hydroxyl propyl cellulose in a pharmaceutical formulation described herein is about 4.8% (w/w). In certain embodiments, the amount of the hydroxyl propyl cellulose in a pharmaceutical formulation described herein is about 4.9% (w/w). In certain embodiments, the amount of the hydroxyl propyl cellulose in a pharmaceutical formulation described herein is about 5.0% (w/w).

In certain embodiments, the amount of the hydroxyl propyl cellulose in a pharmaceutical formulation described herein is about 10 mg to about 20 mg. In certain embodiments, the amount of the hydroxyl propyl cellulose in a pharmaceutical formulation described herein is about 10 mg, about 11 mg, about 12 mg, about 13 mg, about 14 mg, about 15 mg, about 16 mg, about 17 mg, about 18 mg, about 19 mg, or about 20 mg. In certain embodiments, the amount of the hydroxyl propyl cellulose in a pharmaceutical formulation described herein is about 10 mg. In certain embodiments, the amount of the hydroxyl propyl cellulose in a pharmaceutical formulation described herein is about 11 mg. In certain embodiments, the amount of the hydroxyl propyl cellulose in a pharmaceutical formulation described herein is about 12 mg. In certain embodiments, the amount of the hydroxyl propyl cellulose in a pharmaceutical formulation described herein is about 13 mg. In certain embodiments, the amount of the hydroxyl propyl cellulose in a pharmaceutical formulation described herein is about 14 mg. In certain embodiments, the amount of the hydroxyl propyl cellulose in a pharmaceutical formulation described herein is about 15 mg. In certain embodiments, the amount of the hydroxyl propyl cellulose in a pharmaceutical formulation described herein is about 16 mg.

In certain embodiments, the amount of lactose monohydrate in a pharmaceutical formulation described herein is about 8% (w/w) to about 20% (w/w). In certain embodiments, the amount of lactose monohydrate in a pharmaceutical formulation described herein is about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), or about 20% (w/w). In certain embodiments, the amount of lactose monohydrate in a pharmaceutical formulation described herein is about 8% (w/w). In certain embodiments, the amount of lactose monohydrate in a pharmaceutical formulation described herein is about 9% (w/w). In certain embodiments, the amount of lactose monohydrate in a pharmaceutical formulation described herein is about 10% (w/w). In certain embodiments, the amount of lactose monohydrate in a pharmaceutical formulation described herein is about 11% (w/w). In certain embodiments, the amount of lactose monohydrate in a pharmaceutical formulation described herein is about 17% (w/w). In certain embodiments, the amount of lactose monohydrate in a pharmaceutical formulation described herein is about 18% (w/w). In certain embodiments, the amount of lactose monohydrate in a pharmaceutical formulation described herein is about 19% (w/w). In certain embodiments, the amount of lactose monohydrate in a pharmaceutical formulation described herein is about 20% (w/w).

In certain embodiments, the amount of lactose monohydrate in a pharmaceutical formulation described herein is about 20 mg to about 80 mg. In certain embodiments, the amount of lactose monohydrate in a pharmaceutical formulation described herein is about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg. In certain embodiments, the amount of lactose monohydrate in a pharmaceutical formulation described herein is about 20 mg. In certain embodiments, the amount of lactose monohydrate in a pharmaceutical formulation described herein is about 25 mg. In certain embodiments, the amount of lactose monohydrate in a pharmaceutical formulation described herein is about 30 mg. In certain embodiments, the amount of lactose monohydrate in a pharmaceutical formulation described herein is about 35 mg. In certain embodiments, the amount of lactose monohydrate in a pharmaceutical formulation described herein is about 65 mg. In certain embodiments, the amount of lactose monohydrate in a pharmaceutical formulation described herein is about 70 mg. In certain embodiments, the amount of lactose monohydrate in a pharmaceutical formulation described herein is about 75 mg. In certain embodiments, the amount of lactose monohydrate in a pharmaceutical formulation described herein is about 80 mg.

In certain embodiments, the amount of magnesium stearate in a pharmaceutical formulation described herein is about 1% (w/w) to about 3% (w/w). In certain embodiments, the amount of magnesium stearate in a pharmaceutical formulation described herein is about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), or about 3% (w/w). In certain embodiments, the amount of magnesium stearate in a pharmaceutical formulation described herein is about 1% (w/w). In certain embodiments, the amount of magnesium stearate in a pharmaceutical formulation described herein is about 1.1% (w/w). In certain embodiments, the amount of magnesium stearate in a pharmaceutical formulation described herein is about 1.2% (w/w). In certain embodiments, the amount of magnesium stearate in a pharmaceutical formulation described herein is about 1.3% (w/w). In certain embodiments, the amount of magnesium stearate in a pharmaceutical formulation described herein is about 2.7% (w/w). In certain embodiments, the amount of magnesium stearate in a pharmaceutical formulation described herein is about 2.8% (w/w). In certain embodiments, the amount of magnesium stearate in a pharmaceutical formulation described herein is about 2.9% (w/w). In certain embodiments, the amount of magnesium stearate in a pharmaceutical formulation described herein is about 3% (w/w).

In certain embodiments, the amount of the magnesium stearate in a pharmaceutical formulation described herein is about 2 mg to about 10 mg. In certain embodiments, the amount of the magnesium stearate in a pharmaceutical formulation described herein is about 2 mg, about 3 mg, about 4 mg, about 5 mg, about 6 mg, about 7 mg, about 8 mg, about 9 mg, or about 10 mg. In certain embodiments, the amount of the magnesium stearate in a pharmaceutical formulation described herein is about 3 mg. In certain embodiments, the amount of the magnesium stearate in a pharmaceutical formulation described herein is about 4 mg. In certain embodiments, the amount of the magnesium stearate in a pharmaceutical formulation described herein is about 5 mg. In certain embodiments, the amount of the magnesium stearate in a pharmaceutical formulation described herein is about 6 mg. In certain embodiments, the amount of the magnesium stearate in a pharmaceutical formulation described herein is about 7 mg. In certain embodiments, the amount of the magnesium stearate in a pharmaceutical formulation described herein is about 8 mg. In certain embodiments, the amount of the magnesium stearate in a pharmaceutical formulation described herein is about 9 mg.

In certain embodiments, the amount of the microcrystalline cellulose in a pharmaceutical formulation described herein is about 15% (w/w) to about 20% (w/w). In certain embodiments, the amount of the microcrystalline cellulose in a pharmaceutical formulation described herein is about 15% (w/w), about 16% (w/w), about 17% (w/w), about 18% (w/w), about 19% (w/w), or about 20% (w/w). In certain embodiments, the amount of the microcrystalline cellulose in a pharmaceutical formulation described herein is about 15% (w/w). In certain embodiments, the amount of the microcrystalline cellulose in a pharmaceutical formulation described herein is about 16% (w/w). In certain embodiments, the amount of the microcrystalline cellulose in a pharmaceutical formulation described herein is about 17% (w/w). In certain embodiments, the amount of the microcrystalline cellulose in a pharmaceutical formulation described herein is about 18% (w/w). In certain embodiments, the amount of the microcrystalline cellulose in a pharmaceutical formulation described herein is about 19% (w/w). In certain embodiments, the amount of the microcrystalline cellulose in a pharmaceutical formulation described herein is about 20% (w/w).

In certain embodiments, the amount of the microcrystalline cellulose in a pharmaceutical formulation described herein is about 55 mg to about 65 mg. In certain embodiments, the amount of the microcrystalline cellulose in a pharmaceutical formulation described herein is about 55 mg, about 55.5 mg, about 56 mg, about 56.5 mg, about 57 mg, about 57.5 mg, about 58 mg, about 58.5 mg, about 59 mg, about 59.5 mg, about 60 mg, about 60.5 mg, about 61 mg, about 61.5 mg, about 62 mg, about 62.5 mg, about 63 mg, about 63.5 mg, about 64 mg, about 64.5 mg, or about 65 mg. In certain embodiments, the amount of the microcrystalline cellulose in a pharmaceutical formulation described herein is about 60 mg. In certain embodiments, the amount of the microcrystalline cellulose in a pharmaceutical formulation described herein is about 60.5 mg. In certain embodiments, the amount of the microcrystalline cellulose in a pharmaceutical formulation described herein is about 61 mg. In certain embodiments, the amount of the microcrystalline cellulose in a pharmaceutical formulation described herein is about 61.5 mg. In certain embodiments, the amount of the microcrystalline cellulose in a pharmaceutical formulation described herein is about 62 mg. In certain embodiments, the amount of the microcrystalline cellulose in a pharmaceutical formulation described herein is about 62.5 mg. In certain embodiments, the amount of the microcrystalline cellulose in a pharmaceutical formulation described herein is about 63 mg. In certain embodiments, the amount of the microcrystalline cellulose in a pharmaceutical formulation described herein is about 63.5 mg. In certain embodiments, the amount of the microcrystalline cellulose in a pharmaceutical formulation described herein is about 64 mg. In certain embodiments, the amount of the microcrystalline cellulose in a pharmaceutical formulation described herein is about 64.5 mg. In certain embodiments, the amount of the microcrystalline cellulose in a pharmaceutical formulation described herein is about 65 mg.

In certain embodiments, the amount of sodium starch glycolate in a pharmaceutical formulation described herein is about 6.5% (w/w) to about 7.5% (w/w). In certain embodiments, the amount of sodium starch glycolate in a pharmaceutical formulation described herein is about 6.5% (w/w), about 6.6% (w/w), about 6.7% (w/w), about 6.8% (w/w), about 6.9% (w/w), about 7% (w/w), about 7.1% (w/w), about 7.2% (w/w), about 7.3% (w/w), about 7.4% (w/w), or about 7.5% (w/w). In certain embodiments, the amount of sodium starch glycolate in a pharmaceutical formulation described herein is about 6.8% (w/w). In certain embodiments, the amount of sodium starch glycolate in a pharmaceutical formulation described herein is about 6.9% (w/w). In certain embodiments, the amount of sodium starch glycolate in a pharmaceutical formulation described herein is about 7% (w/w). In certain embodiments, the amount of sodium starch glycolate in a pharmaceutical formulation described herein is about 7.1% (w/w). In certain embodiments, the amount of sodium starch glycolate in a pharmaceutical formulation described herein is about 7.2% (w/w). In certain embodiments, the amount of sodium starch glycolate in a pharmaceutical formulation described herein is about 7.3% (w/w).

In certain embodiments, the amount of sodium starch glycolate in a pharmaceutical formulation described herein is about 20 mg to about 30 mg. In certain embodiments, the amount of sodium starch glycolate in a pharmaceutical formulation described herein is about 20 mg, about 21 mg, about 22 mg, about 23 mg, about 24 mg, about 25 mg, about 26 mg, about 27 mg, about 28 mg, about 29 mg, or about 30 mg. In certain embodiments, the amount of sodium starch glycolate in a pharmaceutical formulation described herein is about 22 mg. In certain embodiments, the amount of sodium starch glycolate in a pharmaceutical formulation described herein is about 23 mg. In certain embodiments, the amount of sodium starch glycolate in a pharmaceutical formulation described herein is about 24 mg. In certain embodiments, the amount of sodium starch glycolate in a pharmaceutical formulation described herein is about 25 mg. In certain embodiments, the amount of sodium starch glycolate in a pharmaceutical formulation described herein is about 26 mg. In certain embodiments, the amount of sodium starch glycolate in a pharmaceutical formulation described herein is about 27 mg. In certain embodiments, the amount of sodium starch glycolate in a pharmaceutical formulation described herein is about 28 mg.

In certain embodiments, the amount of the povidone in a pharmaceutical formulation described herein is 0% (w/w) to about 0.5% (w/w). In certain embodiments, the amount of the povidone in a pharmaceutical formulation described herein is about 0.1% (w/w), about 0.2% (w/w), about 0.3% (w/w), about 0.4% (w/w), or about 0.5% (w/w). In certain embodiments, the amount of the povidone in a pharmaceutical formulation described herein is about 0.1% (w/w). In certain embodiments, the amount of the povidone in a pharmaceutical formulation described herein is about 0.2% (w/w). In certain embodiments, the amount of the povidone in a pharmaceutical formulation described herein is about 0.3% (w/w). In certain embodiments, the amount of the povidone in a pharmaceutical formulation described herein is about 0.4% (w/w). In certain embodiments, the amount of the povidone in a pharmaceutical formulation described herein is about 0.5% (w/w).

In certain embodiments, the amount of the povidone in a pharmaceutical formulation described herein is 0 mg to about 1 mg. In certain embodiments, the amount of the povidone in a pharmaceutical formulation described herein is about 0.1 mg, about 0.2 mg, about 0.3 mg, about 0.4 mg, about 0.5 mg, about 0.6 mg, about 0.7 mg, about 0.8 mg, about 0.9 mg, or about 1 mg. In certain embodiments, the amount of the povidone in a pharmaceutical formulation described herein is about 0.6 mg. In certain embodiments, the amount of the povidone in a pharmaceutical formulation described herein is about 0.7 mg. In certain embodiments, the amount of the povidone in a pharmaceutical formulation described herein is about 0.8 mg. In certain embodiments, the amount of the povidone in a pharmaceutical formulation described herein is about 0.9 mg. In certain embodiments, the amount of the povidone in a pharmaceutical formulation described herein is about 1 mg.

In certain embodiments, the amount of sodium lauryl sulfate in a pharmaceutical formulation described herein is about 0% (w/w) to about 0.8% (w/w). In certain embodiments, the amount of sodium lauryl sulfate in a pharmaceutical formulation described herein is about 0.3% (w/w), about 0.4% (w/w), about 0.5% (w/w), about 0.6% (w/w), about 0.7% (w/w), or about 0.8% (w/w). In certain embodiments, the amount of sodium lauryl sulfate in a pharmaceutical formulation described herein is about 0.3% (w/w). In certain embodiments, the amount of sodium lauryl sulfate in a pharmaceutical formulation described herein is about 0.4% (w/w). In certain embodiments, the amount of sodium lauryl sulfate in a pharmaceutical formulation described herein is about 0.5% (w/w). In certain embodiments, the amount of sodium lauryl sulfate in a pharmaceutical formulation described herein is about 0.6% (w/w).

In certain embodiments, the amount of sodium lauryl sulfate in a pharmaceutical formulation described herein is 0 mg to about 2.5 mg. In certain embodiments, the amount of sodium lauryl sulfate in a pharmaceutical formulation described herein is about 1.5 mg, about 1.6 mg, about 1.7 mg, about 1.8 mg, about 1.9 mg, about 2 mg, about 2.1 mg, about 2.2 mg, about 2.3 mg, about 2.4 mg, or about 2.5 mg. In certain embodiments, the amount of sodium lauryl sulfate in a pharmaceutical formulation described herein is about 1.7 mg. In certain embodiments, the amount of sodium lauryl sulfate in a pharmaceutical formulation described herein is about 1.8 mg. In certain embodiments, the amount of sodium lauryl sulfate in a pharmaceutical formulation described herein is about 1.9 mg. In certain embodiments, the amount of sodium lauryl sulfate in a pharmaceutical formulation described herein is about 2 mg. In certain embodiments, the amount of sodium lauryl sulfate in a pharmaceutical formulation described herein is about 2.1 mg. In certain embodiments, the amount of sodium lauryl sulfate in a pharmaceutical formulation described herein is about 2.2 mg.

In various embodiments, provided herein is a pharmaceutical formulation comprising
(i) about 45% (w/w) to about 55% (w/w) bempedoic acid;
(ii) about 1% (w/w) to about 2% (w/w) colloidal silicon dioxide;
(iii) about 3% (w/w) to about 5% (w/w) of a hydroxy propyl cellulose;
(iv) about 8% (w/w) to about 20% (w/w) lactose monohydrate;
(v) about 1% (w/w) to about 3% (w/w) magnesium stearate;
(vi) about 15% (w/w) to about 20% (w/w) a microcrystalline cellulose; and
(vii) about 6.5% (w/w) to about 7.5% (w/w) sodium starch glycolate.

In various embodiments, provided herein is a pharmaceutical formulation comprising
(i) about 45% (w/w) to about 55% (w/w) bempedoic acid;
(ii) about 1% (w/w) to about 2% (w/w) colloidal silicon dioxide;
(iii) about 3% (w/w) to about 5% (w/w) of a hydroxy propyl cellulose;
(iv) about 8% (w/w) to about 20% (w/w) lactose monohydrate;
(v) about 1% (w/w) to about 3% (w/w) magnesium stearate;
(vi) about 15% (w/w) to about 20% (w/w) a microcrystalline cellulose;
(vii) about 6.5% (w/w) to about 7.5% (w/w) sodium starch glycolate;
(viii) 0% (w/w) to about 3% (w/w) ezetimibe;
(ix) 0% (w/w) to about 0.5% (w/w) of a povidone; and
(x) 0% (w/w) to about 0.8% (w/w) sodium lauryl sulfate.

In various embodiments, provided herein is a pharmaceutical formulation comprising
(i) 180 mg bempedoic acid;
(ii) about 3 mg to about 6 mg colloidal silicon dioxide;
(iii) about 10 mg to about 20 mg of a hydroxy propyl cellulose;
(iv) about 20 mg to about 80 mg lactose monohydrate;
(v) about 2 mg to about 10 mg magnesium stearate;
(vi) about 55 mg to about 65 mg a microcrystalline cellulose; and
(vii) about 20 mg to about 30 mg sodium starch glycolate.

In various embodiments, provided herein is a pharmaceutical formulation comprising
(i) 180 mg bempedoic acid;
(ii) about 3 mg to about 6 mg colloidal silicon dioxide;
(iii) about 10 mg to about 20 mg of a hydroxy propyl cellulose;
(iv) about 20 mg to about 80 mg lactose monohydrate;
(v) about 2 mg to about 10 mg magnesium stearate;
(vi) about 55 mg to about 65 mg a microcrystalline cellulose;
(vii) about 20 mg to about 30 mg sodium starch glycolate;
(viii) 0 mg to about 10 mg ezetimibe;
(ix) 0 mg to about 1 mg of a povidone; and
(x) 0 mg to about 2.5 mg sodium lauryl sulfate.

In certain embodiments, the pharmaceutical formulation further comprises a coating. In certain embodiments, the coating comprises Opadry White 85F18422. In certain embodiments, the coating comprises Opadry AMB II Blue.

In certain embodiments, the pharmaceutical formulations described herein may be administered in a unit dosage form and may be prepared by any method well known in the art of pharmacy.

In another aspect, provided herein are solid dosage forms comprising a pharmaceutical formulation described herein. In certain embodiments, the solid dosage forms described herein is to be used for oral administration.

In various embodiments, the pharmaceutical formulations described herein are formulated for oral delivery. In certain embodiments, the pharmaceutical formulations described herein are formulated as an oral dosage form. Examples of oral dosage forms include, but are not limited to a drench, a tablet, a capsule, a softgel capsule, a cachet, a pill, an emulsion, a lozenge, a solution, a suspension, a bolus, a powder, an elixir or syrup, a pastille, a mouthwash, a granule, or a paste for application to the tongue. In some embodiments, the pharmaceutical formulation is formulated as a tablet.

In certain embodiments, a pharmaceutical formulation described herein can be used for the treatment of a disease, disorder, or condition described herein.

### Methods of Use and Treatments

Provided herein are methods of reducing the risk of a major adverse cardiovascular event (MACE) (e.g., cardiovascular death, myocardial infarction, stroke, or coronary revascularization) in an adult at high risk for a cardiovascular disease event (e.g., an adult with established cardiovascular disease or two or more risk factors for cardiovascular disease). Also provided herein are methods of reducing low-density lipoprotein cholesterol (LDL-C) in an adult with primary hyperlipidemia.

### (1) Myocardial Infarction

In one aspect, provided herein are methods of reducing the risk of myocardial infarction in an adult with established cardiovascular disease or two or more risk factors for cardiovascular disease, the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of reducing the risk of myocardial infarction in an adult with established cardiovascular disease or two or more risk factors for cardiovascular disease, the method comprising administering to the adult a pharmaceutical formulation described herein.

In various embodiments, provided herein is a method of reducing the risk of myocardial infarction in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof.

In certain embodiments, the myocardial infarction is nonfatal myocardial infarction.

In various embodiments, provided herein is a method of reducing the risk of non-fatal myocardial infarction in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients.

In certain embodiments, administration of the pharmaceutical formulation reduces the risk of non-fatal myocardial infarction in the adult by about 20%, about 21%, about 22%, about 23%, about 24%, about 25%, about 26%, about 27%, about 28%, about 29%, or about 30% as compared to an adult with established cardiovascular disease or without established cardiovascular disease and at high risk for a cardiovascular disease event administered a placebo.

In certain embodiments, administration of the pharmaceutical formulation reduces the risk of non-fatal myocardial infarction by about 20%, about 21%, about 22%, about 23%, about 24%, about 25%, about 26%, about 27%, about 28%, about 29%, or about 30% as compared to a placebo in a human population with established cardiovascular disease or without established cardiovascular disease and at high risk for a cardiovascular disease event.

In certain embodiments, administration of the pharmaceutical formulation reduces the risk of non-fatal myocardial infarction in the adult by at least about 20%, by at least about 21%, by at least about 22%, by at least about 23%, by at least about 24%, by at least about 25%, by at least about 26%, by at least about 27%, by at least about 28%, by at least about 29%, or by at least about 30% as compared to an adult with established cardiovascular disease or without established cardiovascular disease and at high risk for a cardiovascular disease event administered a placebo.

In certain embodiments, administration of the pharmaceutical formulation reduces the risk of non-fatal myocardial infarction by at least about 20%, by at least about 21%, by at least about 22%, by at least about 23%, by at least about 24%, by at least about 25%, by at least about 26%, by at least about 27%, by at least about 28%, by at least about 29%, or by at least about 30% as compared to a placebo in a human population having established cardiovascular disease or without established cardiovascular disease and at high risk for a cardiovascular disease event.

In various embodiments, provided herein is a method of reducing the risk of non-fatal myocardial infarction in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients, wherein administration of the pharmaceutical formulation reduces the risk of non-fatal myocardial infarction in the adult by about 27% as compared to an adult with established cardiovascular disease or without established cardiovascular disease and at high risk for a cardiovascular disease event administered a placebo or as compared to a placebo in a human population having established cardiovascular disease or without established cardiovascular disease and at high risk for a cardiovascular disease event.

In various embodiments, provided herein is a method of reducing the risk of coronary revascularization in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients.

In certain embodiments, administration of the pharmaceutical formulation reduces the risk of coronary revascularization in the adult by about 15%, about 16%, about 17%, about 18%, about 19%, about 20%, about 21%, about 22%, about 23%, about 24%, or about 25% as compared to an adult with established cardiovascular disease or without established cardiovascular disease and at high risk for a cardiovascular disease event administered a placebo.

In certain embodiments, administration of the pharmaceutical formulation reduces the risk of coronary revascularization in the adult by about 15%, about 16%, about 17%, about 18%, about 19%, about 20%, about 21%, about 22%, about 23%, about 24%, or about 25% as compared to a placebo in a human population having established cardiovascular disease or without established cardiovascular disease and at high risk for a cardiovascular disease event.

In certain embodiments, administration of the pharmaceutical formulation reduces the risk of coronary revascularization in the adult by at least about 15%, by at least about 16%, by at least about 17%, by at least about 18%, by at least about 19%, by at least about 20%, by at least about 21%, by at least about 22%, by at least about 23%, by at least about 24%, or by at least about 25% as compared to an adult with established cardiovascular disease or without established cardiovascular disease and at high risk for a cardiovascular disease event administered a placebo.

In certain embodiments, administration of the pharmaceutical formulation reduces the risk of coronary revascularization in the adult by at least about 15%, by at least about 16%, by at least about 17%, by at least about 18%, by at least about 19%, by at least about 20%, by at least about 21%, by at least about 22%, by at least about 23%, by at least about 24%, or by at least about 25% as compared to a placebo in a human population having established cardiovascular disease or without established cardiovascular disease and at high risk for a cardiovascular disease event.

In various embodiments, provided herein is a method of reducing the risk of coronary revascularization in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients, wherein administration of the pharmaceutical formulation reduces the risk of coronary revascularization in the adult by about 19% as compared to an adult with established cardiovascular disease or without established cardiovascular disease and at high risk for a cardiovascular disease event administered a placebo or as compared to a placebo in a human population having established cardiovascular disease or without established cardiovascular disease and at high risk for a cardiovascular disease event.

In certain embodiments, the adult has established cardiovascular disease.

In certain embodiments, the adult has two or more risk factors for cardiovascular disease. In certain embodiments, the two or more risk factors for cardiovascular disease are selected from the group consisting of history of tobacco use such as cigarette smoking, diabetes mellitus (DM), hypertension, high body mass index (BMI), obesity, and dyslipidemia. Other risk factors can include age, high blood pressure, unhealthy diet, physical inactivity, a coronary calcium score greater than 400 (Agatston score), elevated LDL-C, elevated hsCRP, familial history, unexplained death in 1 and 2 degree family, sudden cardiac death, risk based on scientific methods or calculators (e.g., Reynolds risk score, Score Risk) and the presence of atherosclerosis (as determined, for example, by an angiogram, an ultrasound of the carotid arteries, and/or computed tomography of the coronary arteries).

In certain embodiments, the adult has primary hyperlipidemia. In certain embodiments, the primary hyperlipidemia is heterozygous familial hypercholesterolemia.

In certain embodiments, the pharmaceutical formulation comprises colloidal silicon dioxide; hydroxyl propyl cellulose; lactose monohydrate; magnesium stearate; microcrystalline cellulose; and sodium starch glycolate. In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the pharmaceutical formulation further comprises 10 mg ezetimibe.

In certain embodiments, the method further comprises administering to the adult another low-density lipoprotein cholesterol (LDL-C) lowering therapy (which also can be referred to herein as simply a “LDL-C lowering therapy”).

In certain embodiments, the LDL-C-lowering therapy comprises niacin, a bile acid resin, a fibrate, or a proprotein convertase subtilisin kexin type 9 (PCSK9) inhibitor.

In certain embodiments, the LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering an LDL-C lowering therapy comprises administering to the adult about 10 mg to about 80 mg atorvastatin. In certain embodiments, administering an LDL-C lowering therapy comprises administering to the adult 10 mg to 80 mg atorvastatin.

In certain embodiments, administering an LDL-C lowering therapy comprises administering to the adult about 10 mg, about 20 mg, about 40 mg, or about 80 mg atorvastatin. In certain embodiments, administering an LDL-C lowering therapy comprises administering to the adult 10 mg, 20 mg, 40 mg, or 80 mg atorvastatin. In certain embodiments, administering an LDL-C lowering therapy comprises administering to the adult about 10 mg atorvastatin. In certain embodiments, administering an LDL-C lowering therapy comprises administering to the adult about 20 mg atorvastatin. In certain embodiments, administering an LDL-C lowering therapy comprises administering to the adult about 40 mg atorvastatin. In certain embodiments, administering an LDL-C lowering therapy comprises administering to the adult about 80 mg atorvastatin. In certain embodiments, administering an LDL-C lowering therapy comprises administering to the adult 10 mg atorvastatin. In certain embodiments, administering an LDL-C lowering therapy comprises administering to the adult 20 mg atorvastatin. In certain embodiments, administering an LDL-C lowering therapy comprises administering to the adult 40 mg atorvastatin. In certain embodiments, administering an LDL-C lowering therapy comprises administering to the adult 80 mg atorvastatin.

In certain embodiments, administering an LDL-C lowering therapy comprises administering to the adult about 20 mg to about 80 mg fluvastatin. In certain embodiments, administering an LDL-C lowering therapy comprises administering to the adult 20 mg to 80 mg fluvastatin.

In certain embodiments, administering an LDL-C lowering therapy comprises administering to the adult about 20 mg, about 40 mg, or about 80 mg fluvastatin. In certain embodiments, administering an LDL-C lowering therapy comprises administering to the adult 20 mg, 40 mg, or 80 mg fluvastatin. In certain embodiments, administering an LDL-C lowering therapy comprises administering to the adult about 20 mg fluvastatin. In certain embodiments, administering an LDL-C lowering therapy comprises administering to the adult about 40 mg fluvastatin. In certain embodiments, administering an LDL-C lowering therapy comprises administering to the adult about 80 mg fluvastatin. In certain embodiments, administering an LDL-C lowering therapy comprises administering to the adult 20 mg fluvastatin. In certain embodiments, administering an LDL-C lowering therapy comprises administering to the adult 40 mg fluvastatin. In certain embodiments, administering an LDL-C lowering therapy comprises administering to the adult 80 mg fluvastatin.

In certain embodiments, administering an LDL-C lowering therapy comprises administering to the adult about 20 mg to about 40 mg lovastatin. In certain embodiments, administering an LDL-C lowering therapy comprises administering to the adult 20 mg to 40 mg lovastatin.

In certain embodiments, administering an LDL-C lowering therapy comprises administering to the adult about 20 mg or about 40 mg lovastatin. In certain embodiments, administering an LDL-C lowering therapy comprises administering to the adult 20 mg or 40 mg lovastatin. In certain embodiments, administering an LDL-C lowering therapy comprises administering to the adult about 20 mg lovastatin. In certain embodiments, administering an LDL-C lowering therapy comprises administering to the adult about 40 mg lovastatin. In certain embodiments, administering an LDL-C lowering therapy comprises administering to the adult 20 mg lovastatin. In certain embodiments, administering an LDL-C lowering therapy comprises administering to the adult 40 mg lovastatin.

In certain embodiments, administering an LDL-C lowering therapy comprises administering to the adult about 1 mg to about 4 mg pitavastatin. In certain embodiments, administering an LDL-C lowering therapy comprises administering to the adult 1 mg to 4 mg pitavastatin.

In certain embodiments, administering an LDL-C lowering therapy comprises administering to the adult about 1 mg, about 2 mg, or about 4 mg pitavastatin. In certain embodiments, administering an LDL-C lowering therapy comprises administering to the adult 1 mg, 2 mg, or 4 mg pitavastatin. In certain embodiments, administering an LDL-C lowering therapy comprises administering to the adult about 1 mg pitavastatin. In certain embodiments, administering an LDL-C lowering therapy comprises administering to the adult about 2 mg pitavastatin. In certain embodiments, administering an LDL-C lowering therapy comprises administering to the adult about 4 mg pitavastatin. In certain embodiments, administering an LDL-C lowering therapy comprises administering to the adult 1 mg pitavastatin. In certain embodiments, administering an LDL-C lowering therapy comprises administering to the adult 2 mg pitavastatin. In certain embodiments, administering an LDL-C lowering therapy comprises administering to the adult 4 mg pitavastatin.

In certain embodiments, administering an LDL-C lowering therapy comprises administering to the adult about 10 mg to about 40 mg pravastatin. In certain embodiments, administering an LDL-C lowering therapy comprises administering to the adult 10 mg to 40 mg pravastatin.

In certain embodiments, administering an LDL-C lowering therapy comprises administering to the adult about 10 mg, about 20 mg, or about 40 mg pravastatin. In certain embodiments, administering an LDL-C lowering therapy comprises administering to the adult 10 mg, 20 mg, or 40 mg pravastatin. In certain embodiments, administering an LDL-C lowering therapy comprises administering to the adult about 10 mg pravastatin. In certain embodiments, administering an LDL-C lowering therapy comprises administering to the adult about 20 mg pravastatin. In certain embodiments, administering an LDL-C lowering therapy comprises administering to the adult about 40 mg pravastatin. In certain embodiments, administering an LDL-C lowering therapy comprises administering to the adult 10 mg pravastatin. In certain embodiments, administering an LDL-C lowering therapy comprises administering to the adult 20 mg pravastatin. In certain embodiments, administering an LDL-C lowering therapy comprises administering to the adult 40 mg pravastatin.

In certain embodiments, administering an LDL-C lowering therapy comprises administering to the adult about 5 mg to about 40 mg rosuvastatin. In certain embodiments, administering an LDL-C lowering therapy comprises administering to the adult 5 mg to 40 mg rosuvastatin.

In certain embodiments, administering an LDL-C lowering therapy comprises administering to the adult about 5 mg, about 10 mg, about 20 mg, or about 40 mg rosuvastatin. In certain embodiments, administering an LDL-C lowering therapy comprises administering to the adult 5 mg, 10 mg, 20 mg, or 40 mg rosuvastatin. In certain embodiments, administering an LDL-C lowering therapy comprises administering to the adult about 5 mg rosuvastatin. In certain embodiments, administering an LDL-C lowering therapy comprises administering to the adult about 10 mg rosuvastatin. In certain embodiments, administering an LDL-C lowering therapy comprises administering to the adult about 20 mg rosuvastatin. In certain embodiments, administering an LDL-C lowering therapy comprises administering to the adult about 40 mg rosuvastatin. In certain embodiments, administering an LDL-C lowering therapy comprises administering to the adult 5 mg rosuvastatin. In certain embodiments, administering an LDL-C lowering therapy comprises administering to the adult 10 mg rosuvastatin. In certain embodiments, administering an LDL-C lowering therapy comprises administering to the adult 20 mg rosuvastatin. In certain embodiments, administering an LDL-C lowering therapy comprises administering to the adult 40 mg rosuvastatin.

In certain embodiments, administering an LDL-C lowering therapy comprises administering to the adult about 10 mg to about 20 mg simvastatin. In certain embodiments, administering an LDL-C lowering therapy comprises administering to the adult 10 mg to 20 mg simvastatin.

In certain embodiments, administering an LDL-C lowering therapy comprises administering to the adult about 10 mg or about 20 mg simvastatin. In certain embodiments, administering an LDL-C lowering therapy comprises administering to the adult 10 mg or 20 mg. In certain embodiments, administering an LDL-C lowering therapy comprises administering to the adult about 10 mg simvastatin. In certain embodiments, administering an LDL-C lowering therapy comprises administering to the adult about 20 mg simvastatin. In certain embodiments, administering an LDL-C lowering therapy comprises administering to the adult 10 mg simvastatin. In certain embodiments, administering an LDL-C lowering therapy comprises administering to the adult 20 mg simvastatin.

In certain embodiments, the LDL-C-lowering therapy comprises ezetimibe.

In certain embodiments, administering an LDL-C lowering therapy comprises administering to the adult about 10 mg ezetimibe. In certain embodiments, administering an LDL-C lowering therapy comprises administering to the adult 10 mg ezetimibe. In certain embodiments, administering an LDL-C lowering therapy comprises administering to the adult orally 10 mg ezetimibe. In certain embodiments, administering an LDL-C lowering therapy comprises administering to the adult orally 10 mg ezetimibe once daily.

In certain embodiments, the LDL-C lowering therapy is administered to the adult once, twice, three, four, five, six, or seven times a week. In certain embodiments, the LDL-C lowering therapy is administered to the adult once, twice, three, four, or five times daily. In certain embodiments, the LDL-C lowering therapy is administered to the adult once daily. In certain embodiments, the LDL-C lowering therapy is administered to the adult twice daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (2) Coronary Revascularization

In one aspect, provided herein are methods of reducing the risk of coronary revascularization in an adult with established cardiovascular disease or two or more risk factors for cardiovascular disease, the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of reducing the risk of coronary revascularization in an adult with established cardiovascular disease or two or more risk factors for cardiovascular disease, the method comprising administering to the adult a pharmaceutical formulation described herein.

In various embodiments, provided herein is a method of reducing the risk of coronary revascularization in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof.

In certain embodiments, the adult has established cardiovascular disease.

In certain embodiments, the adult has two or more risk factors for cardiovascular disease. In certain embodiments, the two or more risk factors for cardiovascular disease are selected from the group consisting of history of tobacco use such as cigarette smoking, diabetes mellitus (DM) hypertension, high body mass index (BMI), obesity, and dyslipidemia. Other risk factors can include age, high blood pressure, unhealthy diet, physical inactivity, a coronary calcium score greater than 400 (Agatston score), and the presence of atherosclerosis (as determined, for example, by an angiogram, an ultrasound of the carotid arteries, and/or computed tomography of the coronary arteries).

In certain embodiments, the adult has primary hyperlipidemia. In certain embodiments, the primary hyperlipidemia is heterozygous familial hypercholesterolemia.

In certain embodiments, the pharmaceutical formulation comprises colloidal silicon dioxide; hydroxyl propyl cellulose; lactose monohydrate; magnesium stearate; microcrystalline cellulose; and sodium starch glycolate. In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the pharmaceutical formulation further comprises 10 mg ezetimibe.

In certain embodiments, the method further comprises administering to the adult a low-density lipoprotein cholesterol (LDL-C) lowering therapy.

In certain embodiments, the LDL-C-lowering therapy comprises niacin, a bile acid resin, a fibrate, or a proprotein convertase subtilisin kexin type 9 (PCSK9) inhibitor.

In certain embodiments, the LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering an LDL-C lowering therapy comprises administering to the adult about 10 mg to about 80 mg atorvastatin. In certain embodiments, administering an LDL-C lowering therapy comprises administering to the adult 10 mg to 80 mg atorvastatin.

In certain embodiments, administering an LDL-C lowering therapy comprises administering to the adult about 10 mg, about 20 mg, about 40 mg, or about 80 mg atorvastatin. In certain embodiments, administering an LDL-C lowering therapy comprises administering to the adult 10 mg, 20 mg, 40 mg, or 80 mg atorvastatin. In certain embodiments, administering an LDL-C lowering therapy comprises administering to the adult about 10 mg atorvastatin. In certain embodiments, administering an LDL-C lowering therapy comprises administering to the adult about 20 mg atorvastatin. In certain embodiments, administering an LDL-C lowering therapy comprises administering to the adult about 40 mg atorvastatin. In certain embodiments, administering an LDL-C lowering therapy comprises administering to the adult about 80 mg atorvastatin. In certain embodiments, administering an LDL-C lowering therapy comprises administering to the adult 10 mg atorvastatin. In certain embodiments, administering an LDL-C lowering therapy comprises administering to the adult 20 mg atorvastatin. In certain embodiments, administering an LDL-C lowering therapy comprises administering to the adult 40 mg atorvastatin. In certain embodiments, administering an LDL-C lowering therapy comprises administering to the adult 80 mg atorvastatin.

In certain embodiments, administering an LDL-C lowering therapy comprises administering to the adult about 20 mg to about 80 mg fluvastatin. In certain embodiments, administering an LDL-C lowering therapy comprises administering to the adult 20 mg to 80 mg fluvastatin.

In certain embodiments, administering an LDL-C lowering therapy comprises administering to the adult about 20 mg, about 40 mg, or about 80 mg fluvastatin. In certain embodiments, administering an LDL-C lowering therapy comprises administering to the adult 20 mg, 40 mg, or 80 mg fluvastatin. In certain embodiments, administering an LDL-C lowering therapy comprises administering to the adult about 20 mg fluvastatin. In certain embodiments, administering an LDL-C lowering therapy comprises administering to the adult about 40 mg fluvastatin. In certain embodiments, administering an LDL-C lowering therapy comprises administering to the adult about 80 mg fluvastatin. In certain embodiments, administering an LDL-C lowering therapy comprises administering to the adult 20 mg fluvastatin. In certain embodiments, administering an LDL-C lowering therapy comprises administering to the adult 40 mg fluvastatin. In certain embodiments, administering an LDL-C lowering therapy comprises administering to the adult 80 mg fluvastatin.

In certain embodiments, administering an LDL-C lowering therapy comprises administering to the adult about 20 mg to about 40 mg lovastatin. In certain embodiments, administering an LDL-C lowering therapy comprises administering to the adult 20 mg to 40 mg lovastatin.

In certain embodiments, administering an LDL-C lowering therapy comprises administering to the adult about 20 mg or about 40 mg lovastatin. In certain embodiments, administering an LDL-C lowering therapy comprises administering to the adult 20 mg or 40 mg lovastatin. In certain embodiments, administering an LDL-C lowering therapy comprises administering to the adult about 20 mg lovastatin. In certain embodiments, administering an LDL-C lowering therapy comprises administering to the adult about 40 mg lovastatin. In certain embodiments, administering an LDL-C lowering therapy comprises administering to the adult 20 mg lovastatin. In certain embodiments, administering an LDL-C lowering therapy comprises administering to the adult 40 mg lovastatin.

In certain embodiments, administering an LDL-C lowering therapy comprises administering to the adult about 1 mg to about 4 mg pitavastatin. In certain embodiments, administering an LDL-C lowering therapy comprises administering to the adult 1 mg to 4 mg pitavastatin.

In certain embodiments, administering an LDL-C lowering therapy comprises administering to the adult about 1 mg, about 2 mg, or about 4 mg pitavastatin. In certain embodiments, administering an LDL-C lowering therapy comprises administering to the adult 1 mg, 2 mg, or 4 mg pitavastatin. In certain embodiments, administering an LDL-C lowering therapy comprises administering to the adult about 1 mg pitavastatin. In certain embodiments, administering an LDL-C lowering therapy comprises administering to the adult about 2 mg pitavastatin. In certain embodiments, administering an LDL-C lowering therapy comprises administering to the adult about 4 mg pitavastatin. In certain embodiments, administering an LDL-C lowering therapy comprises administering to the adult 1 mg pitavastatin. In certain embodiments, administering an LDL-C lowering therapy comprises administering to the adult 2 mg pitavastatin. In certain embodiments, administering an LDL-C lowering therapy comprises administering to the adult 4 mg pitavastatin.

In certain embodiments, administering an LDL-C lowering therapy comprises administering to the adult about 10 mg to about 40 mg pravastatin. In certain embodiments, administering an LDL-C lowering therapy comprises administering to the adult 10 mg to 40 mg pravastatin.

In certain embodiments, administering an LDL-C lowering therapy comprises administering to the adult about 10 mg, about 20 mg, or about 40 mg pravastatin. In certain embodiments, administering an LDL-C lowering therapy comprises administering to the adult 10 mg, 20 mg, or 40 mg pravastatin. In certain embodiments, administering an LDL-C lowering therapy comprises administering to the adult about 10 mg pravastatin. In certain embodiments, administering an LDL-C lowering therapy comprises administering to the adult about 20 mg pravastatin. In certain embodiments, administering an LDL-C lowering therapy comprises administering to the adult about 40 mg pravastatin. In certain embodiments, administering an LDL-C lowering therapy comprises administering to the adult 10 mg pravastatin. In certain embodiments, administering an LDL-C lowering therapy comprises administering to the adult 20 mg pravastatin. In certain embodiments, administering an LDL-C lowering therapy comprises administering to the adult 40 mg pravastatin.

In certain embodiments, administering an LDL-C lowering therapy comprises administering to the adult about 5 mg to about 40 mg rosuvastatin. In certain embodiments, administering an LDL-C lowering therapy comprises administering to the adult 5 mg to 40 mg rosuvastatin.

In certain embodiments, administering an LDL-C lowering therapy comprises administering to the adult about 5 mg, about 10 mg, about 20 mg, or about 40 mg rosuvastatin. In certain embodiments, administering an LDL-C lowering therapy comprises administering to the adult 5 mg, 10 mg, 20 mg, or 40 mg rosuvastatin. In certain embodiments, administering an LDL-C lowering therapy comprises administering to the adult about 5 mg rosuvastatin. In certain embodiments, administering an LDL-C lowering therapy comprises administering to the adult about 10 mg rosuvastatin. In certain embodiments, administering an LDL-C lowering therapy comprises administering to the adult about 20 mg rosuvastatin. In certain embodiments, administering an LDL-C lowering therapy comprises administering to the adult about 40 mg rosuvastatin. In certain embodiments, administering an LDL-C lowering therapy comprises administering to the adult 5 mg rosuvastatin. In certain embodiments, administering an LDL-C lowering therapy comprises administering to the adult 10 mg rosuvastatin. In certain embodiments, administering an LDL-C lowering therapy comprises administering to the adult 20 mg rosuvastatin. In certain embodiments, administering an LDL-C lowering therapy comprises administering to the adult 40 mg rosuvastatin.

In certain embodiments, administering an LDL-C lowering therapy comprises administering to the adult about 10 mg to about 20 mg simvastatin. In certain embodiments, administering an LDL-C lowering therapy comprises administering to the adult 10 mg to 20 mg simvastatin.

In certain embodiments, administering an LDL-C lowering therapy comprises administering to the adult about 10 mg or about 20 mg simvastatin. In certain embodiments, administering an LDL-C lowering therapy comprises administering to the adult 10 mg or 20 mg. In certain embodiments, administering an LDL-C lowering therapy comprises administering to the adult about 10 mg simvastatin. In certain embodiments, administering an LDL-C lowering therapy comprises administering to the adult about 20 mg simvastatin. In certain embodiments, administering an LDL-C lowering therapy comprises administering to the adult 10 mg simvastatin. In certain embodiments, administering an LDL-C lowering therapy comprises administering to the adult 20 mg simvastatin.

In certain embodiments, the LDL-C-lowering therapy comprises ezetimibe.

In certain embodiments, administering an LDL-C lowering therapy comprises administering to the adult about 10 mg ezetimibe. In certain embodiments, administering an LDL-C lowering therapy comprises administering to the adult 10 mg ezetimibe.

In certain embodiments, the LDL-C lowering therapy is administered to the adult once, twice, three, four, five, six, or seven times a week. In certain embodiments, the LDL-C lowering therapy is administered to the adult once, twice, three, four, or five times daily. In certain embodiments, the LDL-C lowering therapy is administered to the adult once daily. In certain embodiments, the LDL-C lowering therapy is administered to the adult twice daily.

In certain embodiments, the LDL-C-lowering therapy comprises ezetimibe. In certain embodiments, administering an LDL-C lowering therapy comprises administering to the adult 10 mg ezetimibe. In certain embodiments, administering an LDL-C lowering therapy comprises administering to the adult orally 10 mg ezetimibe. In certain embodiments, administering an LDL-C lowering therapy comprises administering to the adult orally 10 mg ezetimibe once daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (3) Major Adverse Cardiovascular Events

In one aspect, provided herein are methods of treating an adult at risk for a major adverse cardiovascular event (MACE), the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of treating an adult at risk for a MACE, the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 15% as compared to an adult at risk for the MACE administered a placebo, and wherein the MACE is cardiovascular death, non-fatal myocardial infarction, or non-fatal stroke (i.e., MACE-3).

In various embodiments, provided herein is a method of treating an adult at risk for a MACE, the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 13% as compared to an adult at risk for the MACE administered a placebo, and wherein the MACE is cardiovascular death, non-fatal myocardial infarction, non-fatal stroke, or coronary revascularization (i.e., MACE-4).

In certain embodiments, the adult has established cardiovascular disease. In certain embodiments, the adult has a documented history of coronary artery disease, symptomatic peripheral arterial disease, and/or cerebrovascular atherosclerotic disease.

In certain embodiments, the adult is without established cardiovascular disease and at high risk for a cardiovascular disease event.

In certain embodiments, the adult has diabetes mellitus. In certain embodiments, the diabetes mellitus is type 1 diabetes or type 2 diabetes.

In certain embodiments, the adult has a Reynolds Risk score of greater than about 30%. In certain embodiments, the adult has a SCORE risk score of greater than about 7.5%.

In certain embodiments, the adult has two or more risk factors for cardiovascular disease. In certain embodiments, the two or more risk factors for cardiovascular disease are selected from the group consisting of gender, age, smoking status, systolic blood pressure, total cholesterol, high-density lipoprotein (HDL) cholesterol, high sensitivity C-reactive protein (hsCRP), low-density lipoprotein cholesterol (LDL-C), and familial history of cardiovascular disease events.

In various embodiments, provided herein is a method of reducing the risk of a MACE in an adult at high risk for a cardiovascular disease event, the method comprising orally administering daily to the adult a pharmaceutical formulation comprising 180 mg bempedoic acid and one or more pharmaceutically acceptable excipients, wherein the adult has no prior cardiovascular events.

In certain embodiments, the MACE is cardiovascular death, non-fatal myocardial infarction, non-fatal stroke, or coronary revascularization (i.e., MACE-3). In certain embodiments, administration of the pharmaceutical formulation results in an about 2.3% absolute risk reduction in MACE as compared to an adult at high risk for a cardiovascular event administered a placebo.

In certain embodiments, the MACE is cardiovascular death, non-fatal myocardial infarction, or non-fatal stroke (i.e., MACE-4). In certain embodiments, administration of the pharmaceutical formulation results in an about 2.4% absolute risk reduction in MACE as compared to an adult at high risk for a cardiovascular event administered a placebo.

In certain embodiments, administration of the pharmaceutical formulation reduces the risk of myocardial infarction in the adult as compared to an adult at high risk for a cardiovascular event administered a placebo. In certain embodiments, administration of the pharmaceutical formulation reduces the risk of cardiovascular death in the adult as compared to an adult at high risk for a cardiovascular event administered a placebo. In certain embodiments, administration of the pharmaceutical formulation reduces the risk of all-cause mortality in the adult as compared to an adult at high risk for a cardiovascular event administered a placebo.

In certain embodiments, the adult has primary hyperlipidemia. In certain embodiments, the primary hyperlipidemia is heterozygous familial hypercholesterolemia.

In various embodiments, provided herein is a method of treating an adult with diabetes at risk for a MACE, the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 20% as compared to an adult at risk for the MACE administered a placebo, and wherein the MACE is cardiovascular death, non-fatal myocardial infarction, or non-fatal stroke.

In various embodiments, provided herein is a method of treating an adult with diabetes at risk for a primary MACE (e.g., an adult with no prior history of cardiovascular events), the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 35% as compared to an adult at risk for the MACE administered a placebo, wherein the MACE is cardiovascular death, non-fatal myocardial infarction, or non-fatal stroke.

In various embodiments, provided herein is a method of treating an adult with diabetes at risk for a secondary MACE (e.g., an adult with a history of one or more cardiovascular events), the method comprising orally administering daily to the adult a pharmaceutical formulation comprising 180 mg bempedoic acid, and one or more pharmaceutically acceptable excipients, wherein administration of the pharmaceutical formulation reduces the risk of the MACE by at least about 14% as compared to an adult at risk for the MACE administered a placebo, wherein the MACE is cardiovascular death, non-fatal myocardial infarction, or non-fatal stroke.

In various embodiments, provided herein is a method of treating an adult with diabetes at risk for a MACE, the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 17% as compared to an adult at risk for the MACE administered a placebo, and wherein the MACE is cardiovascular death, non-fatal myocardial infarction, non-fatal stroke, or coronary revascularization.

In various embodiments, provided herein is a method of treating an adult with diabetes at risk for a primary MACE (e.g., an adult with no prior history of cardiovascular events), the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 30% as compared to an adult at risk for the MACE administered a placebo, and wherein the MACE is cardiovascular death, non-fatal myocardial infarction, non-fatal stroke, or coronary revascularization.

In various embodiments, provided herein is a method of treating an adult with diabetes at risk for a secondary MACE (e.g., an adult with a history of one or more cardiovascular events), the method comprising orally administering daily to the adult a pharmaceutical formulation comprising 180 mg bempedoic acid, and one or more pharmaceutically acceptable excipients, wherein administration of the pharmaceutical formulation reduces the risk of the MACE about 13% as compared to an adult at risk for the MACE administered a placebo, and wherein the MACE is cardiovascular death, non-fatal myocardial infarction, non-fatal stroke, or coronary revascularization.

In various embodiments, provided herein is a method of reducing the risk of a MACE in an adult with diabetes at high risk for a cardiovascular disease event, the method comprising orally administering daily to the adult a pharmaceutical formulation comprising 180 mg bempedoic acid and one or more pharmaceutically acceptable excipients, wherein the adult has no prior cardiovascular events.

In certain embodiments, the one or more pharmaceutically acceptable excipients is selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof.

In certain embodiments, the pharmaceutical formulation comprises colloidal silicon dioxide; hydroxyl propyl cellulose; lactose monohydrate; magnesium stearate; microcrystalline cellulose; and sodium starch glycolate. In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the other LDL-C-lowering therapy comprises niacin, a bile acid resin, a fibrate, or a proprotein convertase subtilisin kexin type 9 (PCSK9) inhibitor.

In certain embodiments, the other LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering the other LDL-C lowering therapy comprises administering to the adult about 10 mg to about 80 mg atorvastatin. In certain embodiments, administering the other LDL-C lowering therapy comprises administering to the adult 10 mg to 80 mg atorvastatin.

In certain embodiments, administering the other LDL-C lowering therapy comprises administering to the adult about 10 mg, about 20 mg, about 40 mg, or about 80 mg atorvastatin. In certain embodiments, administering the other LDL-C lowering therapy comprises administering to the adult 10 mg, 20 mg, 40 mg, or 80 mg atorvastatin. In certain embodiments, administering the other LDL-C lowering therapy comprises administering to the adult about 10 mg atorvastatin. In certain embodiments, administering the other LDL-C lowering therapy comprises administering to the adult about 20 mg atorvastatin. In certain embodiments, administering the other LDL-C lowering therapy comprises administering to the adult about 40 mg atorvastatin. In certain embodiments, administering the other LDL-C lowering therapy comprises administering to the adult about 80 mg atorvastatin. In certain embodiments, administering the other LDL-C lowering therapy comprises administering to the adult 10 mg atorvastatin. In certain embodiments, administering the other LDL-C lowering therapy comprises administering to the adult 20 mg atorvastatin. In certain embodiments, administering the other LDL-C lowering therapy comprises administering to the adult 40 mg atorvastatin. In certain embodiments, administering the other LDL-C lowering therapy comprises administering to the adult 80 mg atorvastatin.

In certain embodiments, administering the other LDL-C lowering therapy comprises administering to the adult about 20 mg to about 80 mg fluvastatin. In certain embodiments, administering the other LDL-C lowering therapy comprises administering to the adult 20 mg to 80 mg fluvastatin.

In certain embodiments, administering the other LDL-C lowering therapy comprises administering to the adult about 20 mg, about 40 mg, or about 80 mg fluvastatin. In certain embodiments, administering the other LDL-C lowering therapy comprises administering to the adult 20 mg, 40 mg, or 80 mg fluvastatin. In certain embodiments, administering the other LDL-C lowering therapy comprises administering to the adult about 20 mg fluvastatin. In certain embodiments, administering the other LDL-C lowering therapy comprises administering to the adult about 40 mg fluvastatin. In certain embodiments, administering the other LDL-C lowering therapy comprises administering to the adult about 80 mg fluvastatin. In certain embodiments, administering the other LDL-C lowering therapy comprises administering to the adult 20 mg fluvastatin. In certain embodiments, administering the other LDL-C lowering therapy comprises administering to the adult 40 mg fluvastatin. In certain embodiments, administering the other LDL-C lowering therapy comprises administering to the adult 80 mg fluvastatin.

In certain embodiments, administering the other LDL-C lowering therapy comprises administering to the adult about 20 mg to about 40 mg lovastatin. In certain embodiments, administering the other LDL-C lowering therapy comprises administering to the adult 20 mg to 40 mg lovastatin.

In certain embodiments, administering the other LDL-C lowering therapy comprises administering to the adult about 20 mg or about 40 mg lovastatin. In certain embodiments, administering the other LDL-C lowering therapy comprises administering to the adult 20 mg or 40 mg lovastatin. In certain embodiments, administering the other LDL-C lowering therapy comprises administering to the adult about 20 mg lovastatin. In certain embodiments, administering the other LDL-C lowering therapy comprises administering to the adult about 40 mg lovastatin. In certain embodiments, administering the other LDL-C lowering therapy comprises administering to the adult 20 mg lovastatin. In certain embodiments, administering the other LDL-C lowering therapy comprises administering to the adult 40 mg lovastatin.

In certain embodiments, administering the other LDL-C lowering therapy comprises administering to the adult about 1 mg to about 4 mg pitavastatin. In certain embodiments, administering the other LDL-C lowering therapy comprises administering to the adult 1 mg to 4 mg pitavastatin.

In certain embodiments, administering the other LDL-C lowering therapy comprises administering to the adult about 1 mg, about 2 mg, or about 4 mg pitavastatin. In certain embodiments, administering the other LDL-C lowering therapy comprises administering to the adult 1 mg, 2 mg, or 4 mg pitavastatin. In certain embodiments, administering the other LDL-C lowering therapy comprises administering to the adult about 1 mg pitavastatin. In certain embodiments, administering the other LDL-C lowering therapy comprises administering to the adult about 2 mg pitavastatin. In certain embodiments, administering the other LDL-C lowering therapy comprises administering to the adult about 4 mg pitavastatin. In certain embodiments, administering the other LDL-C lowering therapy comprises administering to the adult 1 mg pitavastatin. In certain embodiments, administering the other LDL-C lowering therapy comprises administering to the adult 2 mg pitavastatin. In certain embodiments, administering the other LDL-C lowering therapy comprises administering to the adult 4 mg pitavastatin.

In certain embodiments, administering the other LDL-C lowering therapy comprises administering to the adult about 10 mg to about 40 mg pravastatin. In certain embodiments, administering the other LDL-C lowering therapy comprises administering to the adult 10 mg to 40 mg pravastatin.

In certain embodiments, administering the other LDL-C lowering therapy comprises administering to the adult about 10 mg, about 20 mg, or about 40 mg pravastatin. In certain embodiments, administering the other LDL-C lowering therapy comprises administering to the adult 10 mg, 20 mg, or 40 mg pravastatin. In certain embodiments, administering the other LDL-C lowering therapy comprises administering to the adult about 10 mg pravastatin. In certain embodiments, administering the other LDL-C lowering therapy comprises administering to the adult about 20 mg pravastatin. In certain embodiments, administering the other LDL-C lowering therapy comprises administering to the adult about 40 mg pravastatin. In certain embodiments, administering the other LDL-C lowering therapy comprises administering to the adult 10 mg pravastatin. In certain embodiments, administering the other LDL-C lowering therapy comprises administering to the adult 20 mg pravastatin. In certain embodiments, administering the other LDL-C lowering therapy comprises administering to the adult 40 mg pravastatin.

In certain embodiments, administering the other LDL-C lowering therapy comprises administering to the adult about 5 mg to about 40 mg rosuvastatin. In certain embodiments, administering the other LDL-C lowering therapy comprises administering to the adult 5 mg to 40 mg rosuvastatin.

In certain embodiments, administering the other LDL-C lowering therapy comprises administering to the adult about 5 mg, about 10 mg, about 20 mg, or about 40 mg rosuvastatin. In certain embodiments, administering the other LDL-C lowering therapy comprises administering to the adult 5 mg, 10 mg, 20 mg, or 40 mg rosuvastatin. In certain embodiments, administering the other LDL-C lowering therapy comprises administering to the adult about 5 mg rosuvastatin. In certain embodiments, administering the other LDL-C lowering therapy comprises administering to the adult about 10 mg rosuvastatin. In certain embodiments, administering the other LDL-C lowering therapy comprises administering to the adult about 20 mg rosuvastatin. In certain embodiments, administering the other LDL-C lowering therapy comprises administering to the adult about 40 mg rosuvastatin. In certain embodiments, administering the other LDL-C lowering therapy comprises administering to the adult 5 mg rosuvastatin. In certain embodiments, administering the other LDL-C lowering therapy comprises administering to the adult 10 mg rosuvastatin. In certain embodiments, administering the other LDL-C lowering therapy comprises administering to the adult 20 mg rosuvastatin. In certain embodiments, administering the other LDL-C lowering therapy comprises administering to the adult 40 mg rosuvastatin.

In certain embodiments, administering the other LDL-C lowering therapy comprises administering to the adult about 10 mg to about 20 mg simvastatin. In certain embodiments, administering the other LDL-C lowering therapy comprises administering to the adult 10 mg to 20 mg simvastatin.

In certain embodiments, administering the other LDL-C lowering therapy comprises administering to the adult about 10 mg or about 20 mg simvastatin. In certain embodiments, administering the other LDL-C lowering therapy comprises administering to the adult 10 mg or 20 mg. In certain embodiments, administering the other LDL-C lowering therapy comprises administering to the adult about 10 mg simvastatin. In certain embodiments, administering the other LDL-C lowering therapy comprises administering to the adult about 20 mg simvastatin. In certain embodiments, administering the other LDL-C lowering therapy comprises administering to the adult 10 mg simvastatin. In certain embodiments, administering the other LDL-C lowering therapy comprises administering to the adult 20 mg simvastatin.

In certain embodiments, the LDL-C lowering therapy is administered to the adult once, twice, three, four, five, six, or seven times a week. In certain embodiments, the other LDL-C lowering therapy is administered to the subject once, twice, three, four, or five times daily. In certain embodiments, the other LDL-C lowering therapy is administered to the subject once daily. In certain embodiments, the other LDL-C lowering therapy is administered to the subject twice daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (4) Primary Hyperlipidemia

In one aspect, provided herein are methods of reducing low-density lipoprotein cholesterol (LDL-C) in an adult with primary hyperlipidemia, the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of reducing low-density lipoprotein cholesterol (LDL-C) in an adult with primary hyperlipidemia, the method comprising administering to the adult a pharmaceutical formulation described herein.

In various embodiments, provided herein is a method of reducing low-density lipoprotein cholesterol (LDL-C) in an adult with primary hyperlipidemia, the method comprising administering to the adult a fixed-dose combination described herein.

In various embodiments, provided herein is a method of reducing LDL-C in an adult with primary hyperlipidemia, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof, alone or in combination with another LDL-C-lowering therapy.

In certain embodiments, the pharmaceutical formulation comprises colloidal silicon dioxide; hydroxyl propyl cellulose; lactose monohydrate; magnesium stearate; microcrystalline cellulose; and sodium starch glycolate.

In certain embodiments, the other LDL-C-lowering therapy comprises ezetimibe. In certain embodiments, administering the other LDL-C-lowering therapy comprises administering to the adult 10 mg ezetimibe. In certain embodiments, administering the other LDL-C-lowering therapy comprises administering to the adult orally 10 mg ezetimibe. In certain embodiments, administering the other LDL-C-lowering therapy comprises administering to the adult orally 10 mg ezetimibe once daily.

In certain embodiments, the pharmaceutical formulation further comprises 10 mg ezetimibe.

In various embodiments, provided herein is a method of reducing LDL-C in an adult with primary hyperlipidemia, the method comprising orally administering daily to the adult a pharmaceutical formulation comprising 180 mg bempedoic acid; 10 mg ezetimibe; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, sodium lauryl sulfate, and combinations thereof, alone or in combination with another LDL-C-lowering therapy.

In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the primary hyperlipidemia is heterozygous familial hypercholesterolemia.

In certain embodiments, the other LDL-C-lowering therapy comprises niacin, a bile acid resin, a fibrate, or a proprotein convertase subtilisin kexin type 9 (PCSK9) inhibitor.

In certain embodiments, the other LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering the other LDL-C lowering therapy comprises administering to the adult about 10 mg to about 80 mg atorvastatin. In certain embodiments, administering the other LDL-C lowering therapy comprises administering to the adult 10 mg to 80 mg atorvastatin.

In certain embodiments, administering the other LDL-C lowering therapy comprises administering to the adult about 10 mg, about 20 mg, about 40 mg, or about 80 mg atorvastatin. In certain embodiments, administering the other LDL-C lowering therapy comprises administering to the adult 10 mg, 20 mg, 40 mg, or 80 mg atorvastatin. In certain embodiments, administering the other LDL-C lowering therapy comprises administering to the adult about 10 mg atorvastatin. In certain embodiments, administering the other LDL-C lowering therapy comprises administering to the adult about 20 mg atorvastatin. In certain embodiments, administering the other LDL-C lowering therapy comprises administering to the adult about 40 mg atorvastatin. In certain embodiments, administering the other LDL-C lowering therapy comprises administering to the adult about 80 mg atorvastatin. In certain embodiments, administering the other LDL-C lowering therapy comprises administering to the adult 10 mg atorvastatin. In certain embodiments, administering the other LDL-C lowering therapy comprises administering to the adult 20 mg atorvastatin. In certain embodiments, administering the other LDL-C lowering therapy comprises administering to the adult 40 mg atorvastatin. In certain embodiments, administering the other LDL-C lowering therapy comprises administering to the adult 80 mg atorvastatin.

In certain embodiments, administering the other LDL-C lowering therapy comprises administering to the adult about 20 mg to about 80 mg fluvastatin. In certain embodiments, administering the other LDL-C lowering therapy comprises administering to the adult 20 mg to 80 mg fluvastatin.

In certain embodiments, administering the other LDL-C lowering therapy comprises administering to the adult about 20 mg, about 40 mg, or about 80 mg fluvastatin. In certain embodiments, administering the other LDL-C lowering therapy comprises administering to the adult 20 mg, 40 mg, or 80 mg fluvastatin. In certain embodiments, administering the other LDL-C lowering therapy comprises administering to the adult about 20 mg fluvastatin. In certain embodiments, administering the other LDL-C lowering therapy comprises administering to the adult about 40 mg fluvastatin. In certain embodiments, administering the other LDL-C lowering therapy comprises administering to the adult about 80 mg fluvastatin. In certain embodiments, administering the other LDL-C lowering therapy comprises administering to the adult 20 mg fluvastatin. In certain embodiments, administering the other LDL-C lowering therapy comprises administering to the adult 40 mg fluvastatin. In certain embodiments, administering the other LDL-C lowering therapy comprises administering to the adult 80 mg fluvastatin.

In certain embodiments, administering the other LDL-C lowering therapy comprises administering to the adult about 20 mg to about 40 mg lovastatin. In certain embodiments, administering the other LDL-C lowering therapy comprises administering to the adult 20 mg to 40 mg lovastatin.

In certain embodiments, administering the other LDL-C lowering therapy comprises administering to the adult about 20 mg or about 40 mg lovastatin. In certain embodiments, administering the other LDL-C lowering therapy comprises administering to the adult 20 mg or 40 mg lovastatin. In certain embodiments, administering the other LDL-C lowering therapy comprises administering to the adult about 20 mg lovastatin. In certain embodiments, administering the other LDL-C lowering therapy comprises administering to the adult about 40 mg lovastatin. In certain embodiments, administering the other LDL-C lowering therapy comprises administering to the adult 20 mg lovastatin. In certain embodiments, administering the other LDL-C lowering therapy comprises administering to the adult 40 mg lovastatin.

In certain embodiments, administering the other LDL-C lowering therapy comprises administering to the adult about 1 mg to about 4 mg pitavastatin. In certain embodiments, administering the other LDL-C lowering therapy comprises administering to the adult 1 mg to 4 mg pitavastatin.

In certain embodiments, administering the other LDL-C lowering therapy comprises administering to the adult about 1 mg, about 2 mg, or about 4 mg pitavastatin. In certain embodiments, administering the other LDL-C lowering therapy comprises administering to the adult 1 mg, 2 mg, or 4 mg pitavastatin. In certain embodiments, administering the other LDL-C lowering therapy comprises administering to the adult about 1 mg pitavastatin. In certain embodiments, administering the other LDL-C lowering therapy comprises administering to the adult about 2 mg pitavastatin. In certain embodiments, administering the other LDL-C lowering therapy comprises administering to the adult about 4 mg pitavastatin. In certain embodiments, administering the other LDL-C lowering therapy comprises administering to the adult 1 mg pitavastatin. In certain embodiments, administering the other LDL-C lowering therapy comprises administering to the adult 2 mg pitavastatin. In certain embodiments, administering the other LDL-C lowering therapy comprises administering to the adult 4 mg pitavastatin.

In certain embodiments, administering the other LDL-C lowering therapy comprises administering to the adult about 10 mg to about 40 mg pravastatin. In certain embodiments, administering the other LDL-C lowering therapy comprises administering to the adult 10 mg to 40 mg pravastatin.

In certain embodiments, administering the other LDL-C lowering therapy comprises administering to the adult about 10 mg, about 20 mg, or about 40 mg pravastatin. In certain embodiments, administering the other LDL-C lowering therapy comprises administering to the adult 10 mg, 20 mg, or 40 mg pravastatin. In certain embodiments, administering the other LDL-C lowering therapy comprises administering to the adult about 10 mg pravastatin. In certain embodiments, administering the other LDL-C lowering therapy comprises administering to the adult about 20 mg pravastatin. In certain embodiments, administering the other LDL-C lowering therapy comprises administering to the adult about 40 mg pravastatin. In certain embodiments, administering the other LDL-C lowering therapy comprises administering to the adult 10 mg pravastatin. In certain embodiments, administering the other LDL-C lowering therapy comprises administering to the adult 20 mg pravastatin. In certain embodiments, administering the other LDL-C lowering therapy comprises administering to the adult 40 mg pravastatin.

In certain embodiments, administering the other LDL-C lowering therapy comprises administering to the adult about 5 mg to about 40 mg rosuvastatin. In certain embodiments, administering the other LDL-C lowering therapy comprises administering to the adult 5 mg to 40 mg rosuvastatin.

In certain embodiments, administering the other LDL-C lowering therapy comprises administering to the adult about 5 mg, about 10 mg, about 20 mg, or about 40 mg rosuvastatin. In certain embodiments, administering the other LDL-C lowering therapy comprises administering to the adult 5 mg, 10 mg, 20 mg, or 40 mg rosuvastatin. In certain embodiments, administering the other LDL-C lowering therapy comprises administering to the adult about 5 mg rosuvastatin. In certain embodiments, administering the other LDL-C lowering therapy comprises administering to the adult about 10 mg rosuvastatin. In certain embodiments, administering the other LDL-C lowering therapy comprises administering to the adult about 20 mg rosuvastatin. In certain embodiments, administering the other LDL-C lowering therapy comprises administering to the adult about 40 mg rosuvastatin. In certain embodiments, administering the other LDL-C lowering therapy comprises administering to the adult 5 mg rosuvastatin. In certain embodiments, administering the other LDL-C lowering therapy comprises administering to the adult 10 mg rosuvastatin. In certain embodiments, administering the other LDL-C lowering therapy comprises administering to the adult 20 mg rosuvastatin. In certain embodiments, administering the other LDL-C lowering therapy comprises administering to the adult 40 mg rosuvastatin.

In certain embodiments, administering the other LDL-C lowering therapy comprises administering to the adult about 10 mg to about 20 mg simvastatin. In certain embodiments, administering the other LDL-C lowering therapy comprises administering to the adult 10 mg to 20 mg simvastatin.

In certain embodiments, administering the other LDL-C lowering therapy comprises administering to the adult about 10 mg or about 20 mg simvastatin. In certain embodiments, administering the other LDL-C lowering therapy comprises administering to the adult 10 mg or 20 mg. In certain embodiments, administering the other LDL-C lowering therapy comprises administering to the adult about 10 mg simvastatin. In certain embodiments, administering the other LDL-C lowering therapy comprises administering to the adult about 20 mg simvastatin. In certain embodiments, administering the other LDL-C lowering therapy comprises administering to the adult 10 mg simvastatin. In certain embodiments, administering the other LDL-C lowering therapy comprises administering to the adult 20 mg simvastatin.

In certain embodiments, the LDL-C lowering therapy is administered to the adult once, twice, three, four, five, six, or seven times a week. In certain embodiments, the other LDL-C lowering therapy is administered to the subject once, twice, three, four, or five times daily. In certain embodiments, the other LDL-C lowering therapy is administered to the subject once daily. In certain embodiments, the other LDL-C lowering therapy is administered to the subject twice daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (4) Outcomes

In certain embodiments, the methods described herein decrease low-density lipoprotein cholesterol (LDL-C) in the adult about 21%, as compared to baseline (e.g., prior to initial administration of the effective amount of bempedoic acid).

In certain embodiments, the methods described herein decrease high-sensitivity C-reactive protein (hsCRP) about 21.6%, as compared to baseline (e.g., prior to the initial administration of the effective amount of bempedoic acid).

In certain embodiments, the methods described herein reduce the risk of myocardial infarction in the adult about 23%, as compared to an adult not receiving the effective amount of bempedoic acid therapy (e.g., 180 mg bempedoic acid).

In certain embodiments, the methods described herein reduce the risk of coronary revascularization in the adult about 19%, as compared to an adult not receiving the effective amount of bempedoic acid therapy (e.g., 180 mg bempedoic acid).

In certain embodiments, the methods described herein do not increase the risk of new onset diabetes in an adult without diabetes, as compared to an adult without diabetes not receiving the effective amount of bempedoic acid therapy (e.g., 180 mg bempedoic acid).

In certain embodiments, the methods described herein do not increase HbAlc levels in an adult without diabetes, as compared to an adult without diabetes not receiving the effective amount of bempedoic acid therapy (e.g., 180 mg bempedoic acid).

### EXAMPLES

Below are examples of specific embodiments for carrying out the present disclosure. The examples are offered for illustrative purposes only and are not intended to limit the scope of the present disclosure in any way.

Any terms not directly defined herein shall be understood to have the meanings commonly associated with them as understood within the art of the disclosure. Certain terms are discussed herein to provide additional guidance to the practitioner in describing the compositions, devices, methods, and the like of aspects of the disclosure, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

### Example 1—A Clinical Study of Bempedoic Acid and Cardiovascular Outcomes in Statin-Intolerant Patients

### 1) Methods

### Trial Organization and Oversight

The CLEAR OUTCOMES trial was a randomized, placebo-controlled, double-blind trial that enrolled patients at 1250 sites in 32 countries. An independent Data Monitoring Committee reviewed safety and efficacy data during the trial.

### Trial Population

Patients 18 to 85 years of age were eligible for enrollment if they met either of two criteria for increased cardiovascular risk, a prior cardiovascular event (secondary prevention) or clinical features that placed them at high risk for a cardiovascular event (primary prevention). (Nicholls S. Lincoff A M, Bays H E, et al. Rationale and design of the CLEAR-outcomes trial: Evaluating the effect of bempedoic acid on cardiovascular events in patients with statin intolerance. Am Heart J. 2021 May; 235:104-112) All patients provided written informed consent. Patients had to report statin intolerance due to an adverse effect that started or increased during statin therapy and resolved or improved after statin therapy was discontinued. Patients could be enrolled if they tolerated a very low average daily statin dose (e.g., an average daily dose of rosuvastatin <5 mg, atorvastatin <10 mg, simvastatin <10 mg, lovastatin <20 mg, pravastatin <40 mg, fluvastatin <40 mg, or pitavastatin <2 mg). Other lipid-lowering therapies were permitted, including ezetimibe, niacin, bile acid resins, fibrates, and/or proprotein convertase subtilisin kexin type 9 (PCSK9) inhibitors. Full inclusion and exclusion criteria are provided in Nicholls et al. (Am Heart J. 2021).

### Randomization and Trial Regimen

Eligible patients entered a 4-week run-in period during which patients were treated with single-blind placebo. If patients were intolerant to placebo treatment or if adherence was <80% by tablet count, they were not eligible for randomization. Patients who successfully completed the run-in period were randomly assigned in a 1:1 ratio to receive bempedoic acid at an oral dose of 180 mg or matching placebo, administered daily. Beginning 6 months after enrollment, the central laboratory notified the investigator if the patient's LDL-cholesterol level was ≥25% higher than baseline. These patients were counseled on healthy dietary guidelines and reminded to take all lipid-regulating medications. If repeat testing confirmed that LDL-cholesterol value met the threshold criteria, the provider could adjust the lipid-lowering treatment regimen per standard of care and local guidelines.

### Trial End Points

The primary end point was the first occurrence of a composite of death from cardiovascular causes, nonfatal myocardial infarction (MI), nonfatal stroke, or coronary revascularization. The first key secondary end point was time to the first occurrence of the composite of cardiovascular death, nonfatal stroke, or MI. Further key secondary end points included time to first occurrence of each of the following outcomes tested in hierarchical order, fatal or nonfatal MI, coronary revascularization, fatal or nonfatal stroke, death from cardiovascular causes, and all-cause mortality.

### Statistical Analysis

This was an event-driven trial designed to provide at least 90% power to detect a 15% relative risk reduction for the primary composite end point at an overall two-sided significance level of 0.05. The trial required a minimum of 1620 primary composite events with ≥24 months of follow-up for all patients and at least 810 first key secondary end points. The average treatment duration was estimated to be 42 months and a lost-to-follow-up rate of 1% per year. Assuming a 3.59% annual event rate in the placebo group, enrollment of 12,600 patients was required, subsequently amended to 14,000. There were no interim efficacy analyses. A hierarchical approach was prespecified to evaluate sequentially each of the six key secondary efficacy end points with statistical significance at each step required to test the next hypothesis, thereby preserving the study-wise Type I error rate at 5%. All analyses were based on the intent-to-treat principle. The primary efficacy end point was analyzed using Cox proportional hazards model with treatment as a factor to generate the hazard ratio (HR) and 95% confidence interval (CI). The p-value was obtained from a log-rank test.

### (2) Results

### Randomization, Patient Characteristics and Follow-Up

13970 patients were randomized, 6992 assigned to the bempedoic acid group and 6978 to the placebo group. Baseline characteristics of treatment groups were similar (Table 1). Mean (+SD) age was 65.5±9.0 years, 48.2% of patients were female, 45.6% had diabetes, 69.9% had experienced a prior cardiovascular event, 22.7% were taking a statin and 11.5% taking ezetimibe. Mean LDL-cholesterol was 139.0 mg/dL, mean HDL-cholesterol 49.5 mg/dL, and median triglycerides 159.0 mg/dL. Patients were followed for a median of 40.6 months. Premature discontinuation of study drug occurred in 29.1% of bempedoic acid-treated patients and 31.7% of placebo-treated patients. Complete assessment for the primary end point was available for 95.3% of patients and vital status available for 99.4% of patients.

TABLE 1
Demographic and Baseline Characteristics of Patients

| | Bempedoic Acid | Placebo |
|---|---|---|
| Characteristic | (N = 6992) | (N = 6978) |
| Age (yrs) | 65.5 ± 9.1 | 65.5 ± 8.9 |
| Age, n (%) | | |
| <65 yrs. | 2859 (40.9) | 2907 (41.7) |
| ≥65 to <75 yrs | 3070 (43.9) | 3027 (43.4) |
| ≥75 yrs | 1063 (15.2) | 1044 (15.0) |
| Females, n (%) | 3361 (48.1) | 3379 (48.4) |
| Race, n (%) | | |
| White | 6397 (91.5) | 6335 (90.8) |
| Black or African American | 156 (2.2) | 172 (2.5) |
| Asian | 130 (1.9) | 136 (1.9) |
| American/Mexican Indian or Alaska Native | 240 (3.4) | 247 (3.5) |
| Native Hawaiian or Other Pacific Islander | 20 (0.3) | 17 (0.2) |
| Other | 1 (<0.1) | 1 (<0.1) |
| Multiple | 48 (0.7) | 70 (1.0) |
| Ethnicity, n (%) | | |
| Hispanic or Latino | 1190 (17.0) | 1143 (16.4) |
| Body-mass index, kg/m² | 29.9 ± 5.2 | 30.0 ± 5.2 |
| LDL-Cholesterol mg/dL, mean (SD) | 139.0 (34.9) | 139.0 (35.2) |
| LDL-Cholesterol category, n (%) | | |
| <130 mg/dL | 3074 (44.0) | 3089 (44.3) |
| ≥130 mg/dL to <160 mg/dL | 2213 (31.7) | 2250 (32.2) |
| ≥160 mg/dL | 1705 (24.4) | 1639 (23.5) |
| hsCRP mg/L, median (IQR) | 2.3 (1.2 to 4.5) | 2.3 (1.2 to 4.5) |
| hsCRP category, n (%) | | |
| <2 mg/dL | 3070 (43.9) | 3071 (44.0) |
| ≥2 mg/dL | 3847 (55.0) | 3840 (55.0) |
| Missing | 75 (1.1) | 67 (1.0) |
| HDL-Cholesterol mg/dL, mean (SD) | 49.6 ± 13.3 | 49.4 ± 13.3 |
| Non-HDL-Cholesterol mg/dL, mean (SD) | 173.8 ± 39.5 | 173.9 ± 40.2 |
| Total Cholesterol mg/dL, mean (SD) | 223.5 ± 40.6 | 223.3 ± 41.1 |
| Triglycerides mg/dL, median (IQR) | 159.5 (118.0-216.5) | 158.5 (118.0-215.0) |
| Baseline eGFR mL/min/1.73 m², n (%) | | |
| ≥90 | 1216 (17.4) | 1233 (17.7) |
| ≥60 to <90 | 4322 (61.8) | 4282 (61.4) |
| ≥30 to <60 | 1437 (20.6) | 1444 (20.7) |
| Cardiovascular risk category, n (%) | | |
| Primary prevention | 2100 (30.0) | 2106 (30.2) |
| Secondary prevention | 4892 (70.0) | 4872 (69.8) |
| Coronary artery disease | 3574 (51.1) | 3536 (50.7) |
| Peripheral arterial disease | 794 (11.4) | 830 (11.9) |
| Cerebrovascular atherosclerotic disease | 1027 (14.7) | 1040 (14.9) |
| Glycemic status, n (%) | | |
| No diabetes | 3848 (55.0) | 3749 (53.7) |
| Normoglycemic | 937 (13.4) | 864 (12.4) |
| Prediabetes§ | 2911 (41.6) | 2885 (41.3) |
| Diabetes^{‡} | 3144 (45.0) | 3229 (46.3) |
| Inadequately controlled diabetes† | 1356 (19.4) | 1369 (19.6) |
| Baseline statin use, n (%) | 1601 (22.9) | 1573 (22.5) |
| Baseline ezetimibe use, n (%) | 803 (11.5) | 809 (11.6) |

* To convert the values for cholesterol to millimoles per liter, multiply by 0.02586. To convert the values for triglycerides to millimoles per liter, multiply by 0.01129. LDL-C denotes low density lipoprotein cholesterol, hsCRP high sensitivity C-reactive protein, HDL-C high density lipoprotein cholesterol.
§Patients at baseline with no medical history of type 2 diabetes, no prior use of glucose-lowering medication, and a HbA1C measurement of 5.7% to 6.4%, or 1 or more measurements of fasting glucose of 100 mg/dL (5.6 mmol/L) to 125 mg/dL (6.9 mmol/L), but not more than 1 value of fasting glucose ≥126 mg/dL (7.0 mmol/L).
^{‡}At baseline, medical history of type 2 diabetes, prior glucose-lowering medication, HbA1C measurement ≥6.5%, or two or more measurements of fasting glucose ≥126 mg/dL (7.0 mmol/L).
†Patients with type 2 diabetes and an HbA1C of 7.0% or greater at baseline.

### Effect on LDL-Cholesterol and High-Sensitivity C-Reactive Protein

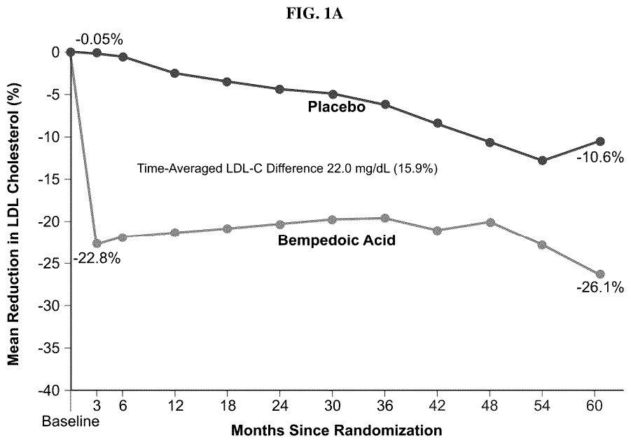

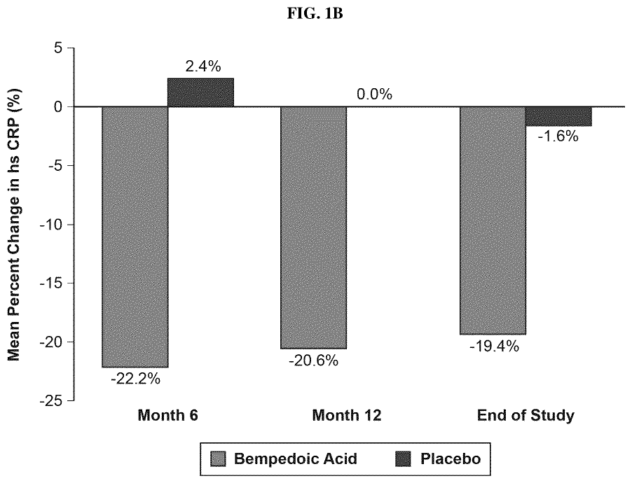

FIG. 1A shows the effect of the trial regimens over time on LDL-cholesterol and Panel B shows the effect on high-sensitivity C-reactive protein (hsCRP). From a baseline mean LDL-cholesterol of 139.0 mg/dL, placebo-subtracted reduction in the bempedoic acid group after 6 months of treatment was 29.2 mg/dL (21.1%) (95% confidence interval [CI]-21.9 to −20.3%) and time-averaged reduction 22.0 mg/dL (15.9%). Among placebo patients, 15.6% crossed over to additional lipid-lowering therapy, compared with 9.4% of bempedoic acid patients. From a median baseline value of 2.3 mg/L, after 6 months treatment, bempedoic acid reduced hsCRP by 21.6% (95% CI −23.7% to −19.6%). Effects of trial regimens on tertiary lipid biomarkers are provided in Table 2.

TABLE 2
Selected Tertiary Lipid Endpoints

| | Bempedoic Acid | Placebo |
|---|---|---|
| Lipid or Biomarker | (N = 6992) | (N = 6978) |
| | Observed mean ± SD | |
| | or median (IQR) | |
| Percent change from baseline | −17.2% ± 21.2 | −0.4% ± 21.4 |
| to Month 6 in non-HDL-C | | |
| Percent change from baseline | −17.1% ± 22.0 | −2.0% ± 22.3 |
| to Month 12 in non-HDL-C | | |
| Percent change from baseline | −16.2% ± 24.3 | −3.8% ± 24.5 |
| to Month 24 in non-HDL-C | | |
| Percent change from baseline | −16.9% ± 16.7 | −0.57% ± 16.3 |
| to Month 6 in total cholesterol | | |
| Percent change from baseline | −14.8% ± 17.3 | −1.8% ± 17.1 |
| to Month 12 in total cholesterol | | |
| Percent change from baseline | −5.8% ± 18.0 | 0.66% ± 15.1 |
| to Month 6 in HDL-C | | |
| Percent change from baseline | −5.7% ± 18.9 | 0.70% ± 16.2 |
| to Month 12 in HDL-C | | |
| Percent change from baseline | −3.4% (−23.7% | −1.7% (−19.5% |
| to Month 6 in triglycerides | to 22.1%) | to 19.5%) |
| Percent change from baseline | −4.8% (−24.8% | −2.3% (−20.8% |
| to Month 12 in triglycerides | to 21.6%) | to 20.4%) |
| Change from baseline to | | |
| Month 6 in HbA1c (%) | | |
| All patients | −0.003 ± 0.6 | 0.05 ± 0.7 |
| Inadequately Controlled | −0.20 ± 1.0 | −0.14 ± 1.2 |
| Diabetes at Baseline | | |
| Change from baseline to | | |
| Month 12 in HbA1c (%) | | |
| All patients | 0.04 ± 0.74 | 0.06 ± 0.7 |
| Inadequately Controlled | −0.06 ± 1.3 | −0.03 ± 1.2 |
| Diabetes at Baseline | | |
| Change from baseline to | | |
| Month 6 in fasting glucose | | |
| (mg/dL) | | |
| All patients | −1.0 (−9.0 | 0.5 (−7.5 |
| | to 7.0) | to 8.5) |
| Inadequately Controlled | −4.0 (−32.0 | 0.5 (−26.5 |
| Diabetes at Baseline | to 25.0) | to 29.0) |
| Change from baseline to | | |
| Month 12 in fasting glucose | | |
| (mg/dL) | | |
| All patients | 0.0 (−8.0 | 1.0 (−7.0 |
| | to 8.5) | to 10.0) |
| Inadequately Controlled | −2.75 (−32.5 | 0.5 (−28.3 |
| Diabetes at Baseline | to 30.5) | to 29.0) |

### Efficacy Endpoints

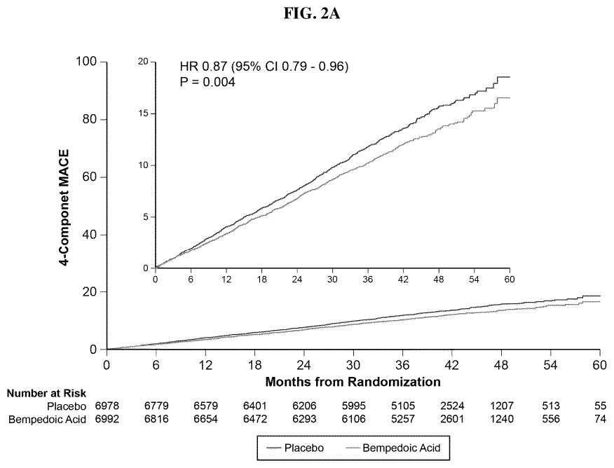

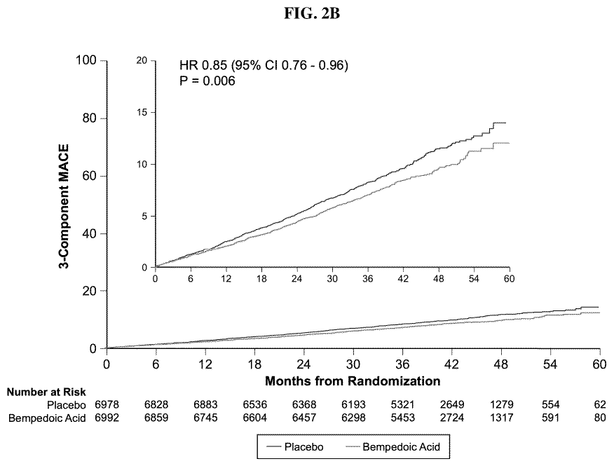

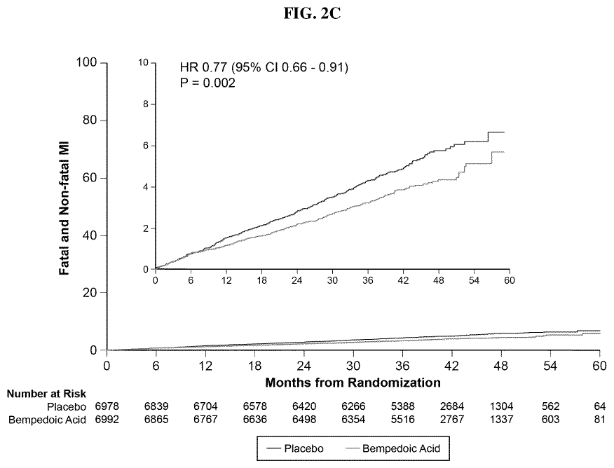

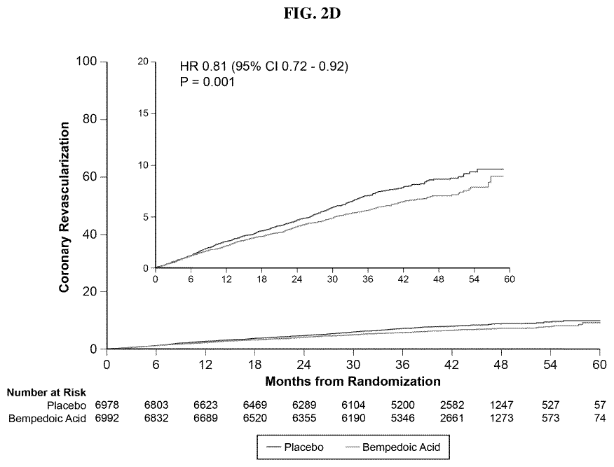

The primary composite end point occurred in 819 patients (11.7%) in the bempedoic acid group and 927 patients (13.3%) in the placebo group HR, 0.87 (95% CI 0.79 to 0.96), P=0.004. (Table 3 and FIG. 2A) Bempedoic acid significantly reduced the risk of the first key secondary composite end point, time to death from cardiovascular causes, nonfatal MI, or stroke, which occurred in 575 patients (8.2%) in the bempedoic acid group and in 663 patients (9.5%) in the placebo group, HR 0.85, 95% CI 0.76 to 0.96, P=0.006. (Table 3 and FIG. 2B) The second key secondary end point, time to first occurrence of fatal or nonfatal MI occurred in 261 (3.7%) of patients in the bempedoic acid treatment group and 334 (4.8%) of patients in the placebo group, HR 0.77 (95% CI 0.66 to 0.91), P=0.002. (Table 3 and FIG. 2C) The third key secondary end point, time to coronary revascularization, occurred in 435 (6.2%) patients in the bempedoic acid group compared with 529 (7.6%) patients in the placebo group, HR 0.81, 95% CI 0.72 to 0.92, P=0.001. (Table 3 and FIG. 2D) Compared with placebo, there were no significant effects of bempedoic acid on the other key secondary end points, time to first occurrence of stroke, death from cardiovascular causes, and all-cause mortality. (Table 3).

Results for the primary end point in prespecified subgroups were determined. There was no statistically significant heterogeneity for any subgroup except for a lower HR in the primary compared with the secondary prevention population, HR 0.68 vs. 0.91 (interaction P=0.03).

TABLE 3
Time to Event Efficacy End Points for the Bempedoic
Acid Treatment Group Compared with Placebo

| | Bempedoic | | | |
|---|---|---|---|---|
| | Acid | Placebo | Hazard | |
| | (N = | (N = | Ratio | P |
| Outcome | 6992) | 6978) | (95% CI) | Value |

number of patients (%)

| | | | | |
|---|---|---|---|---|
| Primary efficacy | 819 | 927 | 0.87 | 0.004 |
| endpoint (MACE-4)* | (11.7) | (13.3) | (0.79-0.96) | |

Key secondary efficacy endpoints

| | | | | |
|---|---|---|---|---|
| 3-component MACE^{§} | 575 | 663 | 0.85 | 0.006 |
| | (8.2) | (9.5) | (0.76-0.96) | |
| Fatal and non- | 261 | 334 | 0.77 | 0.002 |
| fatal MI | (3.7) | (4.8) | (0.66-0.91) | |
| Coronary | 435 | 529 | 0.81 | 0.001 |
| revascularization | (6.2) | (7.6) | (0.72-0.92 | |
| Fatal and non-fatal | 135 | 158 | 0.85 | NA† |
| stroke† | (1.9) | (2.3) | (0.67-1.07) | |
| Death from | 269 | 257 | 1.04 | NA† |
| cardiovascular causes† | (3.8) | (3.7) | (0.88-1.24) | |
| All-cause mortality† | 434 | 420 | 1.03 | NA† |
| | (6.2) | (6.0) | (0.90-1.18) | |

Additional secondary time-to-event end points

| | | | | |
|---|---|---|---|---|
| All-cause mortality, | 962 | 1062 | 0.89 | NA† |
| nonfatal MI, nonfatal | (13.8) | (15.2) | (0.82-0.97) | |
| stroke, or coronary | | | | |
| revascularization | | | | |
| 5 component MACE^{‡} | 831 | 952 | 0.86 | NA† |
| | (11.9) | (13.6) | (0.78-0.94) | |
| Non-fatal MI | 236 | 317 | 0.73 | NA† |
| | (3.4) | (4.5) | (0.62-0.87 | |
| Fatal MI | 29 | 21 | 1.38 | NA† |
| | (0.4) | (0.3) | (0.79-2.42) | |
| Non-fatal stroke | 119 | 144 | 0.82 | NA† |
| | (1.7) | (2.1) | (0.64-1.05) | |
| Fatal stroke | 18 | 16 | 1.12 | NA† |
| | (0.3) | (0.2) | (0.57-2.20) | |
| Fatal and nonfatal | 118 | 150 | 0.78 | NA† |
| non-hemorrhagic | (1.7) | (2.1) | (0.61-0.99) | |
| stroke | | | | |
| Fatal and nonfatal | 20 | 9 | 2.21 | NA† |
| hemorrhagic stroke | (0.3) | (0.1) | (1.01-4.85) | |
| Hospitalization for | 91 | 137 | 0.66 | NA† |
| unstable angina | (1.3) | (2.0) | (0.50-0.86) | |
| New-onset type 2 | 429/3848 | 433/3749 | 0.95 | NA† |
| diabetes mellitus^{#} | (11.1) | (11.5) | (0.83-1.09) | |

Change in secondary lipid and biomarker efficacy end points

| | Mean or Median | | Difference | |
|---|---|---|---|---|
| | (95% CI) | | (95% CI) | |
| Mean change from | −21.1% | −0.8% | −20.3% | NA† |
| baseline to month | (−21.6 | (−1.4% | (−21.1% | |
| 6 in mean LDL-C^{€} | to −20.5%) | to −0.2%) | to −19.5%) | |
| Median change from | −22.2% | 2.4% | −21.6% | NA† |
| baseline to month | (−23.5 | (0 to | (−23.7 | |
| 6 in hsCRP | to −20.8%) | 4.2%) | to −19.6%) | |
| Change from baseline | −0.04% | −0.01% | −0.03% | NA† |
| to month 12 in HbA1C^{€} | (−0.12 | (−0.09 | (−0.14 | |
| in patients with | to 0.03) | to 0.06) | to 0.08) | |
| inadequately controlled | | | | |
| type 2 diabetes mellitus^{£} | | | | |

MACE denotes major adverse cardiovascular events, MI myocardial infarction, CI confidence intervals, LDL-C low density lipoprotein cholesterol, hsCRP high-sensitivity C-reactive protein.
†As prespecified in the hierarchical testing procedure, all p values after the first nonsignificant p value are not presented.
*The primary efficacy endpoint (MACE-4) is the time to first occurrence of an adjudicated event for a composite that includes death from cardiovascular causes, nonfatal MI, nonfatal stroke, or coronary revascularization.
^{§}Time to first occurrence of the composite endpoint of death from cardiovascular causes, nonfatal MI, or nonfatal stroke (MACE-3).
^{‡}Time to first occurrence of death from cardiovascular causes, nonfatal MI, nonfatal stroke, coronary revascularization, or hospitalization for unstable angina (MACE-5).
^{#}Glycated hemoglobin level ≥6.5% or two or more measurements of fasting glucose ≥126 mg/dL (7.0 mmol/L) in patients with baseline glycemic status of no diabetes.
^{€}Results adjusted for baseline LDL-C or HbA1c values, based on a pattern-mixed model for missing data.
^{£}Defined as diabetes and a glycated hemoglobin level of 7% or greater at baseline.

### Adverse Effects

Adverse events are reported in Table 4. There were not clinically meaningful between group differences in overall rates of adverse events, serious adverse events, or adverse events leading to drug discontinuation. Investigator-reported prespecified adverse events of special interest were balanced except for more frequent hepatic enzyme elevations (4.5% vs 3.0%) and renal impairment (11.5% vs. 8.6%) in the bempedoic acid group. Musculoskeletal adverse events occurred in 15.0% of bempedoic acid treated patients and 15.4% of placebo patients. Rates of liver transaminase elevations >3×ULN were more frequent in the bempedoic acid group. Mean changes in creatinine and uric acid from baseline were larger for bempedoic acid compared with placebo. A higher incidence of hyperuricemia (10.9% vs 5.6%), gout (3.1% vs 2.1%) and cholelithiasis (2.2% vs 1.2%) occurred in the bempedoic acid group.

TABLE 4
Adverse Events and Safety Laboratory Findings

| | Bempedoic | |
|---|---|---|
| | Acid | Placebo |
| | (N = 7001), | (N = 6964), |
| | n (%) | n (%) |
| Any treatment emergent | 6041 (86.3) | 5921 (85.0) |
| adverse event | | |
| Serious treatment | 1778 (25.4) | 1743 (25.0) |
| emergent adverse event | | |
| Adverse event leading | 768 (11.0) | 726 (10.4) |
| to drug discontinuation | | |

Prespecified Adverse Events of Special Interest

| | | |
|---|---|---|
| Any muscle disorder | 1052 (15.0) | 1070 (15.4) |
| Myalgia | 393 (5.6) | 471 (6.8) |
| Discontinuation of treatment | 124 (1.8) | 129 (1.9) |
| due to myalgia | | |
| New onset diabetes in patients | 621/3856 (16.1) | 640/3740 (17.1) |
| without diabetes at baseline | | |
| New onset diabetes in patients | 569/2918 (19.5) | 586/2877 (20.4) |
| with pre-diabetes at baseline^{#} | | |
| New onset diabetes in patients | 52/938 (5.5) | 54/863 (6.3) |
| with normoglycemia at | | |
| baseline^{#} | | |
| Worsening hyperglycemia* | 713/3145 (22.7) | 746/3224 (23.1) |
| Hypoglycemia | 304 (4.3) | 267 (3.8) |
| Metabolic acidosis | 13 (0.2) | 11 (0.2) |
| Elevated hepatic enzymes | 317 (4.5) | 209 (3.0) |
| Renal impairment | 802 (11.5) | 599 (8.6) |
| Neurocognitive disorders | 58 (0.8) | 69 (1.0) |
| Atrial fibrillation | 229 (3.3) | 246 (3.5) |
| Adjudicated tendon rupture | 86 (1.2) | 66 (0.9) |
| Tendinopathies | 118 (1.7) | 128 (1.8) |
| Malignancies | 329 (4.7) | 345 (5.0) |

Other Adverse Events

| | | |
|---|---|---|
| Hyperuricemia | 763 (10.9) | 393 (5.6) |
| Gout | 215 (3.1) | 143 (2.1) |
| Cholelithiasis | 152 (2.2) | 81 (1.2) |

Laboratory Results after 6 months, mg/dL

| | | |
|---|---|---|
| Change from baseline in | 0.76 ± 1.2 | −0.03 ± 1.0 |
| uric acid levels | | |
| Change from baseline in | 0.05 ± 0.2 | 0.01 ± 0.2 |
| creatinine levels | | |

Laboratory Results after 12 months

| | | |
|---|---|---|
| Change from baseline in | 0.04 ± 0.74 | 0.06 ± 0.70 |
| glycated hemoglobin level | | |
| (%)^{@} | | |

Enzyme abnormalities at any visit

| | | |
|---|---|---|
| Creatine kinase levels >5x | 45 (0.6) | 40 (0.6) |
| ULN (single occurrence) | | |
| Creatine kinase levels >5x | 8 (0.1) | 8 (0.1) |
| ULN (repeated and | | |
| confirmed) | | |
| Alanine aminotransferase | 83 (1.2) | 53 (0.8) |
| level >3 times ULN^{&} | | |
| Aspartate aminotransferase | 80 (1.1) | 43 (0.6) |
| level >3 times ULN^{&} | | |

ULN denotes upper limit of normal
*In patients with diabetes at baseline
^{#}Patients with prediabetes at baseline were defined as: No past medical history of diabetes and with glycated hemoglobin level of ≥5.7% and <6.5% or 1 or more measurement of fasting glucose ≥100 mg/dL (5.6 mmol/L), but not more than 1 value of fasting glucose ≥126 mg/dL (7.0 mmol/L). Patients with normoglycemia at baseline did not meet criteria for prediabetes.
^{@}Not a prespecified safety measure
^{&}Repeated and confirmed

### Discussion

In primary and secondary prevention patients unable or unwilling to take guideline-recommended doses of statins, treatment with bempedoic acid resulted in a significant 13% reduction in the primary composite end point of death from cardiovascular causes, stroke, MI, or coronary revascularization. Hierarchical testing of the first three key secondary end points also showed significant benefits for bempedoic acid treatment. The composite of death from cardiovascular causes, stroke or MI was reduced by 15%, the incidence of fatal or nonfatal MI reduced by 23%, and coronary revascularization reduced by 19%. After 6 months treatment, bempedoic acid, compared with placebo, reduced LDL-cholesterol by a mean of 29.2 mg/dL (21.1%) and hsCRP by a median of 21.6%.

Bempedoic acid treatment was well tolerated with rates of discontinuation for any reason, including musculoskeletal complaints, similar to placebo (Table 4). There were no differences for other prespecified adverse events of special interest, including new onset or worsening of diabetes mellitus, hypoglycemia and metabolic acidosis, neurocognitive disorders, atrial fibrillation, tendinopathies including tendon rupture and malignancies. A higher incidence of cholelithiasis was reported.

The similar incidence of muscle-related complaints in the bempedoic acid and placebo groups confirms the utility of bempedoic acid as an alternative LDL-cholesterol lowering therapy in patients unable or unwilling to take statins (Table 4).

The time-averaged reduction in LDL-cholesterol of 22.0 mg/dL (0.57 mmol/L) would be expected to result in approximately the reduction observed in cardiovascular events (FIG. 1A).

In conclusion, in primary and secondary prevention patients unable to tolerate recommended doses of statins, bempedoic acid lowered LDL-cholesterol and hsCRP and significantly reduced the primary composite end point, time to death from cardiovascular causes, nonfatal MI, nonfatal stroke, or coronary revascularization by 13%. The narrower key secondary end point that excluded revascularization was reduced by 15% and the incidence of fatal or nonfatal MI was reduced by 23%.

### Example 2—Bempedoic Acid for Primary Prevention of Cardiovascular Events in Statin-Intolerant Patients

### 1) Methods

### Trial Organization and Oversight

The rationale, design and methods of the CLEAR Outcomes trial is described in Example 1. The trial was conducted at 1250 sites in 32 countries and designed by the Cleveland Clinic Coordinating Center for Clinical Research (C5Research) and an academic Executive Committee in collaboration with the sponsor. The protocol was approved by ethics committees at participating sites and all patients provided written informed consent before enrollment. A contract research organization collected the data which were transferred to C5Research at completion of the trial whose statisticians conducted the analyses described herein. An independent Data Monitoring Committee monitored the trial.

### Trial Population

Primary prevention patients 18 to 85 years of age with an LDL cholesterol ≥100 mg/dL with clinical features placing them at high risk for a first cardiovascular event were eligible. Criteria for high cardiovascular risk included risk scores, such as the Reynolds Risk Score and the SCORE project (Ridker et al., Development and validation of improved algorithms for the assessment of global cardiovascular risk in women: the Reynolds Risk Score, JAMA. 2007 Feb. 14; 297(6):611-9; Ridker et al., C-reactive protein and parental history improve global cardiovascular risk prediction. The Reynolds Risk Score for men, Circulation. 2008; 118: 2243-2251; European Guidelines on CVD Prevention in Clinical Practice. 2016. Eur J Prev Cardiol. 2016 July; 23(11):NP1-NP96), a coronary artery calcium score >400 Agatston units (AU) or presence of either type 1 or 2 diabetes in women aged >65 years or men older than 60 years.

Investigators were responsible for assessing the patient for statin intolerance. Patients had to report statin intolerance due to an adverse effect that started or increased during statin therapy and resolved or improved after statin therapy was discontinued. Entry criteria required inability to tolerate 2 or more statins at any dose or 1 statin and unwillingness to attempt a second statin or advised by a physician not to attempt taking a second statin. Both the patient and site investigator were required to provide written confirmation that the patient was statin intolerant and aware of the benefits of statins to reduce the risk of cardiovascular events, including death, and acknowledge that many patients who are unable to tolerate a statin can tolerate a different statin or dose. Patients could be enrolled if they tolerated a very low average daily statin dose defined as rosuvastatin <5 mg, atorvastatin <10 mg, simvastatin <10 mg, lovastatin <20 mg, pravastatin <40 mg, fluvastatin <40 mg, or pitavastatin <2 mg. Other background lipid-lowering therapies were permitted, including ezetimibe, niacin, bile acid resins, fibrates, and/or proprotein convertase subtilisin kexin type 9 (PCSK9) inhibitors.

### Study Procedures

Eligible patients entered a 4-week run-in period during which patients were treated with single-blind placebo. If patients were intolerant to placebo treatment or if adherence was <80% by tablet count, they were not eligible for randomization. Patients who successfully completed the run-in period were randomly assigned in a 1:1 ratio to receive a 180 mg oral dose of bempedoic acid or matching placebo, administered daily. During the trial, LDL cholesterol and hsCRP results were masked. Beginning 6 months after randomization, the central laboratory notified the investigator, who remained blinded to treatment assignment, if the patient's LDL cholesterol level was ≥25% higher than baseline. Repeat testing was performed after the patient was counseled about diet and encouraged to take all medications as prescribed. The patient returned for a repeat fasting blood lipid profile to confirm whether the LDL cholesterol value exceeded the threshold criteria. If confirmed, the patient's treatment regimen for LDL cholesterol lowering could be adjusted, per standard of care and local practice.

### Study End Points

The primary end point was the time to first occurrence of a composite of death from cardiovascular causes, nonfatal myocardial infarction (MI), nonfatal stroke, or coronary revascularization (4-component MACE). Key secondary end points included the following: 1) time to the first occurrence of a composite of cardiovascular death, nonfatal stroke, or nonfatal MI (3-component MACE); 2) fatal or nonfatal MI; 3) coronary revascularization; 4) fatal or nonfatal stroke; 5) cardiovascular death; and 6) and all-cause mortality. Additional adjudicated time-to-event end points included hospitalization for unstable angina and a 5-component composite that included cardiovascular death, nonfatal MI, nonfatal stroke, coronary revascularization, and hospitalization for unstable angina. End points were adjudicated by C5Research Clinical Endpoints Committee personnel blinded to treatment assignments.

### Statistical Analysis

The efficacy end points were analyzed using Cox proportional hazards model with treatment as a factor to generate the hazard ratio (HR) and 95% confidence interval (CI). P-values were obtained from a log-rank test (two-sided). The proportional hazards assumption was evaluated based on Schoenfeld residuals. Significance testing was performed using 2-sided tests (α=0.05). All efficacy outcomes were adjusted for baseline characteristics including geographic region, age, gender, race, ethnicity, LDL cholesterol, body mass index, hsCRP, estimated glomerular filtration rate, use of any lipid-modifying therapy at baseline, and diabetes status (diabetes, pre-diabetes, normoglycemia). Changes in lipids were calculated using least-square (LS) means adjusted for baseline. Hodges-Lehmann estimate of location shift was used to summarize changes in hsCRP and triglycerides. Baseline data for two patients with missing BMI were imputed for efficacy models using the average measurement of the overall cohort. The statistical analysis plan prespecified subgroup analyses for 4-component MACE and 3-component MACE. Therefore, P values are provided only for 4 component and 3-component MACE. Efficacy analyses included all randomized patients based on the intent-to-treat principle. Outcomes were not adjusted for multiplicity. Adverse event summaries were based on the safety population that included all patients who underwent randomization and received at least one dose of study drug.

### 2) Results

### Study Population

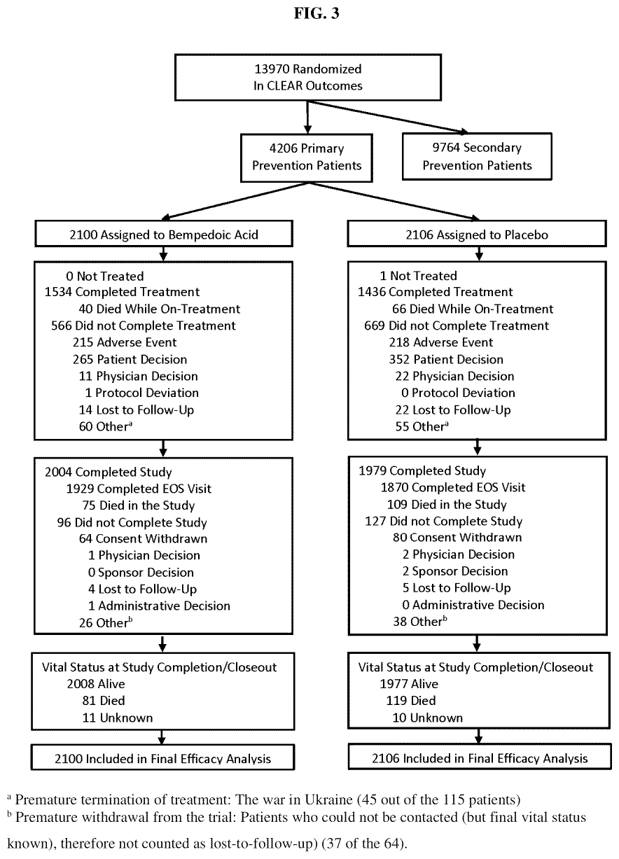

Between December 2016 and August 2019, 22084 patients were screened and 14016 were randomized with 13970 included in the full analysis after exclusion of 46 participants from a site determined to have created fraudulent patients. Within the full analysis set, 4206 patients (30%) met primary prevention criteria, 2100 randomized to the bempedoic acid treatment group and 2106 to the placebo group. For the pooled treatment groups, mean (SD) age was 67.9 (6.8) years, 59.0% of patients were female, 66.1% had diabetes, 19.3% were taking a statin and 8.0% ezetimibe. Mean LDL cholesterol was 142.5 mg/dL, mean HDL-cholesterol 51.0 mg/dL, median triglycerides 161.8 mg/dL and median hsCRP 2.4 mg/L. These baseline characteristics were similar in both treatment groups (Table 5). High risk primary prevention patients were followed for a median of 39.9 months. Complete assessment for the primary end point was available for 94.7% and vital status available for 99.5% of these patients. Efficacy end points at sites in Ukraine were censored after the start of the conflict on Feb. 24, 2022. The flow of patients through the trial is shown in FIG. 3.

TABLE 5
Demographic and Baseline Characteristics of Patients^{a}

| | Bempedoic Acid | Placebo |
|---|---|---|
| Characteristic | (N = 2100) | (N = 2106) |
| Age, yrs (SD) | 67.9 (6.9) | 68.0 (6.8) |
| Females, n (%) | 1234 (58.8) | 1247 (59.2) |
| Males, n (%) | 866 (41.2) | 859 (40.8) |
| Race, n (%)^{a} | 1936 (92.2) | 1913 (90.8) |
| American/Mexican Indian | 49 (2.3) | 49 (2.3) |
| or Alaska Native | | |
| Black or African American | 66 (3.1) | 67 (3.2) |
| Native Hawaiian or Pacific | 8 (0.4) | 6 (0.3) |
| Islander | | |
| Other | 0 | 1 |
| White, n (%) | 1936 (92.2) | 1913 (90.8) |
| Ethnicity, n (%)^{a} | | |
| Hispanic or Latino, | 399 (19.0) | 378 (17.9) |
| Not Hispanic or Latino | 1791 (81.0%) | 1728 (82.1%) |
| Body-mass index, kg/m² mean | 30.2 (5.3) | 30.4 (5.4) |
| (SD) | | |
| Body Mass Index >35 kg/m², | 350 (16.7%) | 367 (17.4%) |
| n (%)_{—} | | |
| Systolic blood pressure, | 135.6 (13.8) | 136.0 (13.6) |
| mmHg mean (SD) | | |
| Systolic BP >140 mm Hg, | 729 (34.7%) | 750 (35.6%) |
| n (%) | | |
| LDL Cholesterol mg/dL, | 142.2 (34.5) | 142.7 (35.9) |
| mean (SD) | | |
| HDL-Cholesterol mg/dL, | 51.1 (13.5) | 50.9 (13.7) |
| mean (SD) | | |
| Non-HDL-Cholesterol mg/dL, | 177.4 (38.7) | 178.2 (41.2) |
| mean (SD) | | |
| Total Cholesterol mg/dL, | 228.5 (40.2) | 229.1 (42.3) |
| mean (SD) | | |
| Triglycerides mg/dL, | 162.0 (120.5- | 161.5 (123.5- |
| median (IQR) | 216.5) | 215.5) |
| hsCRP mg/L, median (IQR) | 2.4 (1.2-4.5) | 2.4 (1.2-4.6) |
| Baseline eGFR mL/min/1.73 | 73.8 (17.3) | 73.2 (17.8) |
| m², mean (SD) | | |
| Cardiovascular risks, | | |
| n (%) | | |
| Diabetes^{b} | 1369 (65.2) | 1412 (67.0) |
| Inadequately controlled | 569(27.1) | 593 (28.2) |
| diabetes^{c} | | |
| Hypertension | 1853 (88.2) | 1854 (88.0) |
| Chronic kidney disease | 146 (7.0) | 155 (7.4) |
| Criteria for increased | | |
| risk, n (%) | | |
| Reynolds Risk Score >30% | 868 (41.3) | 922 (43.8) |
| or a SCORE Risk Score | | |
| of >7.5% over 10 years^{d} | | |
| Coronary artery calcium | 86 (4.1) | 55 (2.6) |
| score >400 AU | | |
| Patients with self-reported | 1150 (54.8) | 1187 (56.4) |
| type 1 or 2 diabetes, aged >65 | | |
| (women) or >60 years (men) | | |
| Region, n (%) | | |
| Eastern Europe | 1114 (53.0) | 1117 (53.0) |
| North America | 446 (21.2) | 439 (20.8) |
| Latin America | 280 (13.3) | 257 (12.2) |
| Western Europe | 168 (8.0) | 180 (8.5) |
| Other^{e} | 92 (4.3) | 113 (5.4) |
| Baseline statin use, n (%)^{f} | 394 (18.8) | 417 (19.8) |
| Baseline ezetimibe use, | 184 (8.8) | 151 (7.2) |
| n (%) | | |

Abbrev.: LDL-C, low density lipoprotein cholesterol; hsCRP, high-sensitivity C-reactive protein; HDL-C, high density lipoprotein cholesterol; SCORE, Systematic Coronary Risk Evaluation; AU, Agatston Units.
^{a}Race and Ethnicity were self-reported. Participants could select more than one category.
^{b}At baseline, medical history of type 2 diabetes, prior glucose-lowering medication, HbA1C measurement ≥6.5%, or two or more measurements of fasting glucose ≥126 mg/dL (7.0 mmol/L).
^{c}Patients with type 2 diabetes and an HbA1C of 7.0% or greater at baseline.
^{d}The REYNOLDS and SCORE risk calculators are described in Ridker et al., JAMA (2013), Ridker et al. Circulation (2008), and Eur. J. Prev. Cardiol. (2016).
^{e}Other regions include Australia, India, New Zealand, South Africa, and Turkey.
^{f}Patients could be enrolled if they were taking doses of a statin less than the lowest approved dose SI conversion factors. To convert the values for cholesterol to millimoles per liter, multiply by 0.02586. To convert the values for triglycerides to millimoles per liter, multiply by 0.01129.

### Effects on LDL Cholesterol and High-Sensitivity C-Reactive Protein

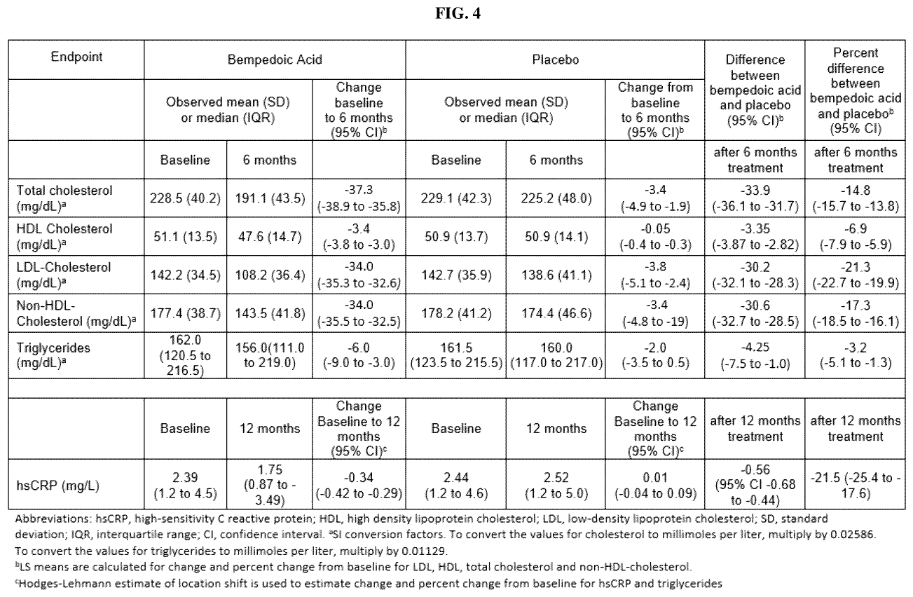

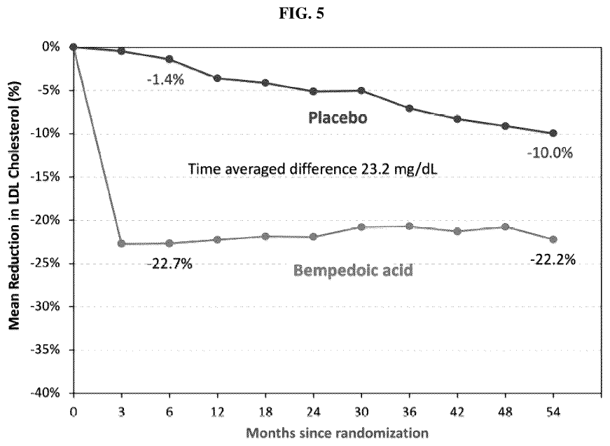

The effects of trial regimens on lipid parameters after 6 months of treatment and hsCRP after 12 months treatment are reported in FIG. 4. From a baseline mean LDL cholesterol of 142.2 mg/dL, LS mean reduction in the bempedoic acid group compared with placebo after 6 months of treatment was 30.2 mg/dL, 95% confidence interval [CI]-32.1 to −28.3 mg/dL, a LS mean difference of 21.3%. With administration of additional lipid modifying therapies to 12.4% of patients in placebo group and 6.7% in the bempedoic acid group (Table 6), the time-averaged difference in LDL cholesterol in the primary prevention subgroup over the course of the trial was 23.2 mg/dL (FIG. 5). From a median baseline value of 2.4 mg/L, after 12 months treatment, bempedoic acid reduced hsCRP by 0.34 mg/L (95% CI −0.42 to −0.29) compared with a reduction of 0.01 mg/L (95% CI −0.04 to 0.09) for placebo, a difference of 0.56 mg/L (21.5%).

TABLE 6
Addition of other lipid modulating therapies during the trial

| | Bempedoic Acid | Placebo | Total |
|---|---|---|---|
| | (N = 2100) | (N = 2106) | (N = 4206) |
| | N (%) | N (%) | N (%) |
| Patients Receiving | 141 (6.7) | 261 (12.4) | 402 (9.6) |
| Adjunctive Lipid | | | |
| Modulating Therapies^{a} | | | |
| Statins | 64 (3.0) | 132 (6.3) | 196 (4.7) |
| Ezetimibe | 41 (2.0) | 106 (5.0) | 147 (3.5) |
| Bile Acid Sequestrants | 4 (0.2) | 3 (0.1) | 7 (0.2) |
| Fibrates | 24 (1.1) | 27 (1.3) | 51 (1.2) |
| PCSK9 inhibitors^{b} | 27 (1.3) | 27 (1.3) | 54 (1.3) |
| PCSK9-mAb | 26 (1.2) | 27 (1.3) | 53 (1.3) |
| PCSK9-siRNA | 1 (<0.1) | 0 | 1 (<0.1) |
| Niacin derivatives | 2 (0.1) | 4 (0.2) | 6 (0.1) |
| Bempedoic Acid | 2 (0.1) | 3 (0.1) | 5 (0.1) |

Addition of Statins post-Randomization

| | | | |
|---|---|---|---|
| Patients who Cross-In | 64 (3.0) | 132 (6.3) | 196 (4.7) |
| to Statin Treatment^{c} | | | |
| Post-Baseline Statin | | | |
| Intensity | | | |
| Very Low | 19 (0.9) | 31 (1.5) | 50 (1.2) |
| Low | 2 (0.1) | 0 | 2 (<0.1) |
| Moderate | 29 (1.4) | 62 (2.9) | 91 (2.2) |
| High | 14 (0.7) | 39 (1.9) | 53 (1.3) |

Abbreviations: Mab, monoclonal antibody;
SiRNA, short interfering RNA
^{a}Non-statin oral prescription lipid modulating therapies (bile acid sequestrants, fibrates, niacin derivatives, ezetimibe, bempedoic acid, bempedoic acid and ezetimibe fixed dose combination that were not usedatbaseline, started after randomization and continued for 90 days or more.
^{b}PCSK9 inhibitor started after randomization, not used at baseline (for evolocumab), or with increased intensity if used at baseline (specifically, for alirocumab switching from 75 mg once every two weeks to 150 mg once every two weeks or 300 mg once every 4 weeks), and continued for 90 days or more if mAb (evolocumab or alirocumab); or ≥1 dose for siRNA (inclisiran) after randomization.
^{c}Any statin, regardless of the specific drug, started after randomization, with increased intensity if used at baseline, and continued for 90 days or more.

### Clinical End Points

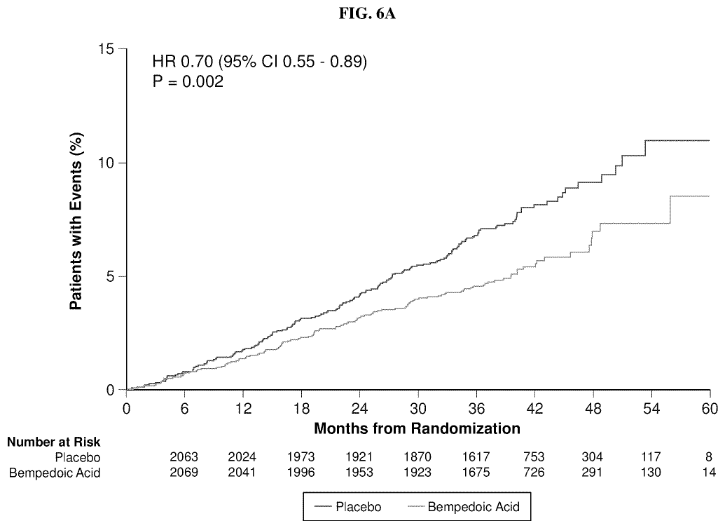

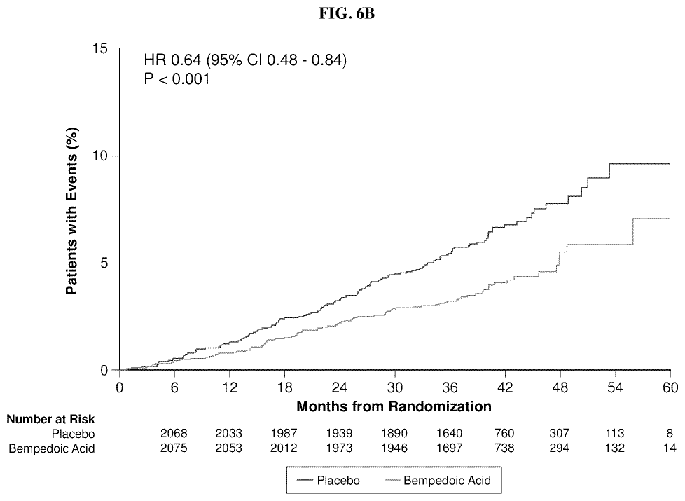

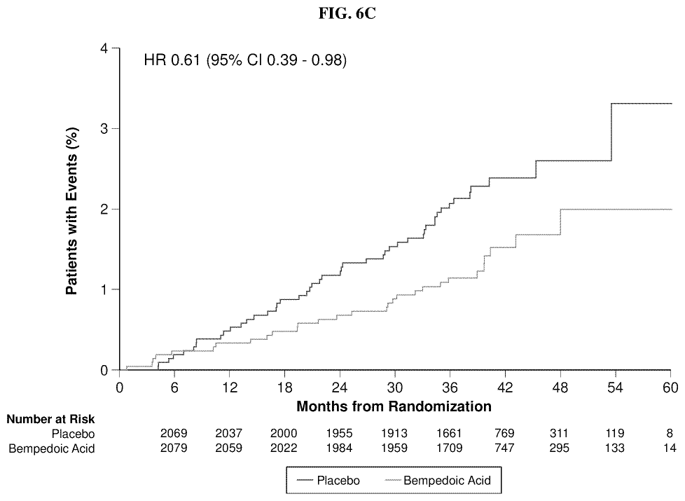

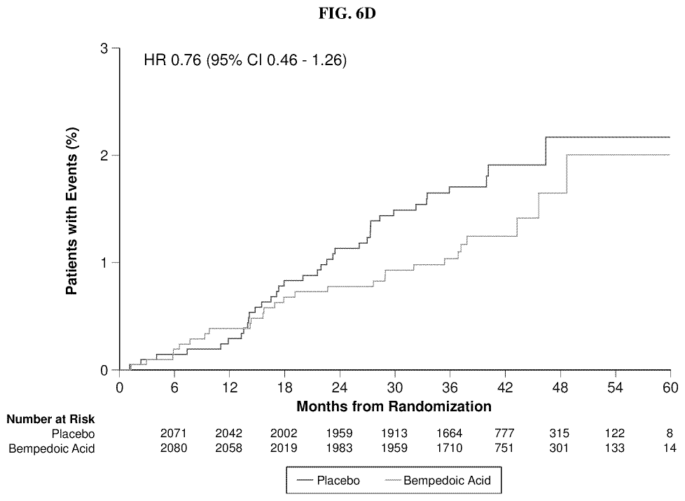

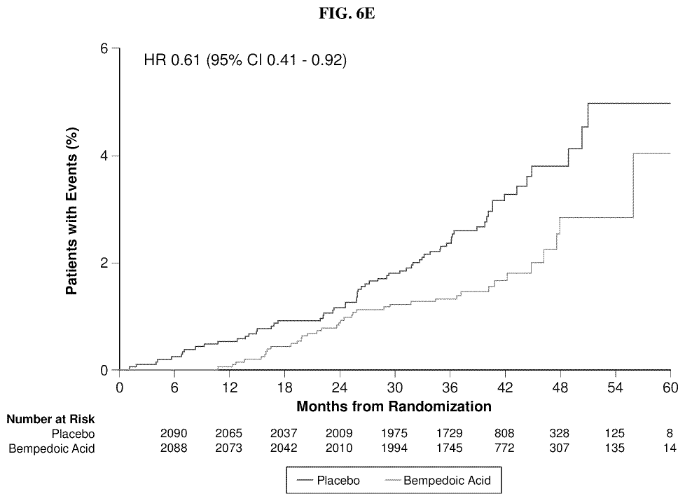

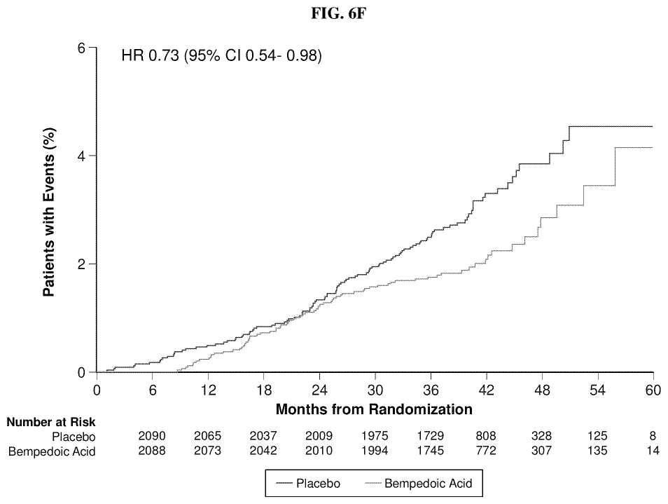

The primary 4-component composite end point occurred in 111 patients (5.3%) in the bempedoic acid group and 161 patients (7.6%) in the placebo group HR 0.70 (95% CI 0.55 to 0.89), P=0.002 (Table 7 and FIG. 6A). The number need to treat (NNT) to prevent one primary composite outcome was 43 patients. Bempedoic acid was associated with a significant reduction in the risk of the 3-component composite end point of time to cardiovascular death, nonfatal MI or stroke, which occurred in 83 patients (4.0%) in the bempedoic acid group and 134 patients (6.4%) in the placebo group, HR 0.64, 95% CI 0.48 to 0.84, P<0.001 (Table 7 and FIG. 6B). Fatal or nonfatal MI occurred in 29 patients (1.4%) in the bempedoic acid group and 47 patients (2.2%) in the placebo group, adjusted HR of 0.61 (95% CI 0.39-0.98) (Table 7 and FIG. 6C). Fatal or nonfatal stroke occurred in 27 patients (1.3%) in the bempedoic acid group and 37 patients (1.8%) in the placebo group, adjusted HR of 0.76 (95% CI 0.46-1.26) (Table 7 and FIG. 6D). Cardiovascular death occurred in 37 patients (1.8%) in the bempedoic acid group and 65 (3.1%) patients in the placebo group, adjusted HR 0.61, 95% CI 0.41 to 0.92 (Table 7 and FIG. 6E). All-cause mortality occurred in 75 patients (3.6%) in the bempedoic acid group and 109 (5.2%) patients in the placebo group, adjusted HR 0.73, 95% CI 0.54 to 0.98 (Table 7 and FIG. 6F).

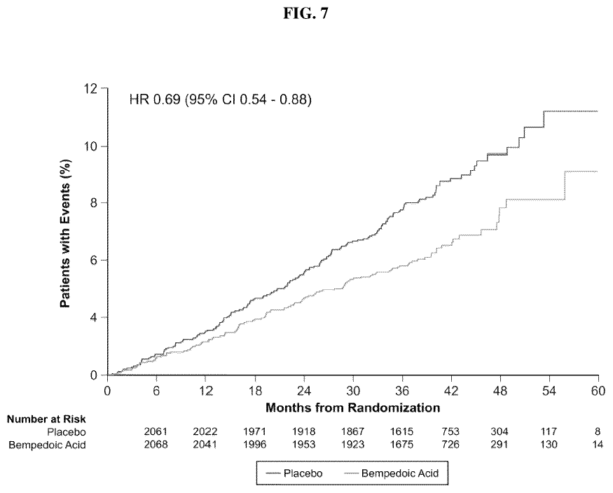

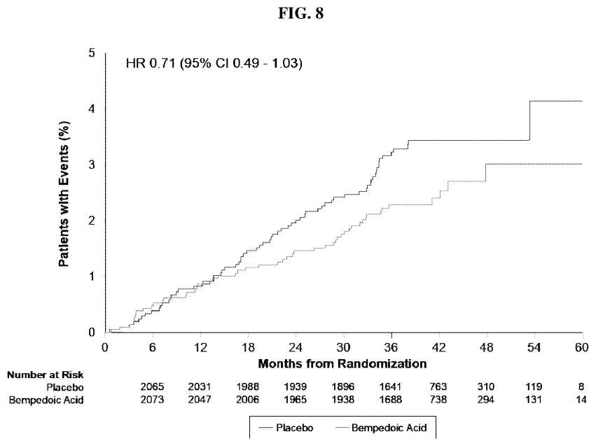

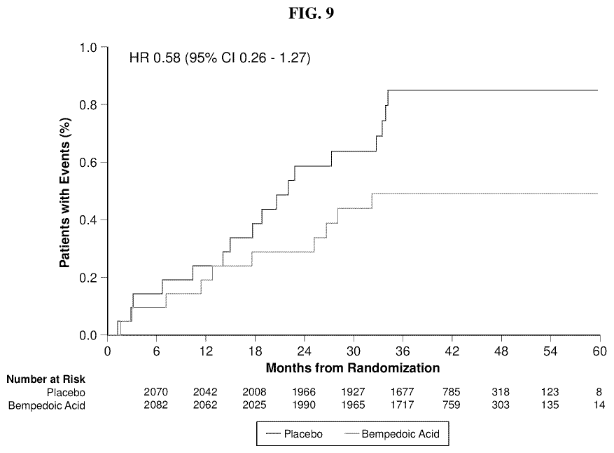

The first occurrence of the 5-component composite of death from cardiovascular causes, MI, stroke, coronary revascularization or hospitalization for unstable angina occurred in 112 patients (5.3%) in the bempedoic acid group and 164 patients (7.8%) in the placebo group, HR 0.69 (95% CI 0.54 to 0.88) (Table 7 and FIG. 7). Coronary revascularization occurred in 50 patients (2.4%) in the bempedoic acid group and 68 patients (3.2%) in the placebo group, HR 0.71 (95% CI 0.49-1.03) (Table 7 and FIG. 8). Hospitalization for unstable angina occurred in 10 patients (0.5%) in the bempedoic acid group and 17 patients (0.8%) in the placebo group, HR 0.58 (95% CI 0.26-1.27) (Table 7 and FIG. 9). Unadjusted analyses of the primary and secondary endpoints showed similar results to the adjusted analyses (Table 8).

TABLE 7
Time to Event Efficacy End Points for the Bempedoic Acid Treatment Group Compared with Placebo

| | Bempedoic Acid | Placebo | | |
|---|---|---|---|---|
| | (N = 2100) | (N = 2106) | Hazard | |
| | 6898 person- | 6807 person- | Ratio^{a} | P |
| Outcome | years follow up | years follow up | (95% CI) | Value^{b} |
| | number of patients (%) | | | |
| Primary efficacy endpoint (MACE-4)^{c} | 111(5.3) | 161 (7.6) | 0.70 (0.55-0.89) | 0.002 |

Secondary efficacy end points

| | | | | |
|---|---|---|---|---|
| 3-component MACE^{d} | 83 (4.0) | 134 (6.4) | 0.64 (0.48-0.84) | <0.001 |
| 5-component MACE^{e} | 112 (5.3) | 164 (7.8) | 0.69 (0.54-0.88) | |

End point Components

| | | | |
|---|---|---|---|
| All-cause mortality | 75 (3.6) | 109 (5.2) | 0.73 (0.54-0.98) |
| Cardiovascular death | 37 (1.8) | 65 (3.1) | 0.61 (0.41-0.92) |
| Fatal and non-fatal MI | 29 (1.4) | 47 (2.2) | 0.61 (0.39-0.98) |
| Fatal and non-fatal stroke | 27(1.3) | 37 (1.8) | 0.76 (0.46-1.26) |
| Coronary revascularization | 50 (2.4) | 68 (3.2) | 0.71 (0.49-1.03) |
| Hospitalization for unstable angina | 10 (0.5) | 17 (0.8) | 0.58 (0.26-1.27) |

Abbreviations: major adverse cardiovascular events, MACE; myocardial infarction, MI; CI, confidence intervals.
^{a}Hazard ratios adjusted for baseline characteristics including geographic region, age, gender, race, ethnicity, baseline LDL cholesterol, body mass index, high-sensitivity C reactive protein, estimated glomerular filtration rate, use of any lipid-modifying therapy at baseline, and diabetes status (diabetes, pre-diabetes, normoglycemia).
^{b}P value is from the log-rank test
^{c}The primary efficacy endpoint (MACE-4) is the time to first occurrence of an adjudicated event for a composite that includes death from cardiovascular causes, nonfatal MI, nonfatal stroke, or coronary revascularization.
^{d}Time to first occurrence of the composite endpoint of death from cardiovascular causes, nonfatal MI, or nonfatal stroke (3 component MACE).
^{e}Time to first occurrence of death from cardiovascular causes, nonfatal MI, nonfatal stroke, coronary revascularization, or hospitalization for unstable angina (MACE-5).

TABLE 8
Unadjusted time to event efficacy end points for the
bempedoic acid treatment group compared with placebo

| | Bempedoic | Placebo | Hazard Ratio^{a} | P |
|---|---|---|---|---|
| | Acid 00) | (N = 2106) | (95% CI) | Value^{a} |
| | number of patients (%) | | | |
| Primary efficacy endpoint (MACE-4)^{b} | 111(5.3) | 161 (7.6) | 0.68 (0.53-0.87) | 0.002 |

Secondary efficacy end points

| | | | | |
|---|---|---|---|---|
| 3-component MACE^{c} | 83 (4.0) | 134 (6.4) | 0.61 (0.46-0.80) | <0.001 |
| 5-component MACE^{d} | 112 (5.3) | 164 (7.8) | 0.67 (0.53-0.86) | |

End point Components

| | | | |
|---|---|---|---|
| All-cause mortality | 75 (3.6) | 109 (5.2) | 0.69 (0.51-0.92) |
| Death from cardiovascular causes | 37 (1.8) | 65 (3.1) | 0.57 (0.38-0.85) |
| Fatal and non-fatal MI | 29 (1.4) | 47 (2.2) | 0.61 (0.38-0.97) |
| Fatal and non-fatal stroke | 27(1.3) | 37 (1.8) | 0.72 (0.44-1.19) |
| Coronary revascularization | 50 (2.4) | 68 (3.2) | 0.73 (0.50-1.05) |
| Hospitalization for unstable angina | 10 (0.5) | 17 (0.8) | 0.58 (0.27-1.27) |

Abbreviations: major adverse cardiovascular events, MACE; myocardial infarction, MI; confidence intervals, CI.
^{a}P value is from the log-rank test.
^{b}The primary efficacy endpoint (MACE-4) is the time to first occurrence of an adjudicated event for a composite that includes death from cardiovascular causes, nonfatal MI, nonfatal stroke, or coronary revascularization.
^{c}Time to first occurrence of the composite endpoint of death from cardiovascular causes, nonfatal MI, or nonfatal stroke (3 component MACE).
^{d}Time to first occurrence of death from cardiovascular causes, nonfatal MI, nonfatal stroke, coronary revascularization, or hospitalization for unstable angina (MACE-5).

### Adverse Events

Adverse events are reported in Table 9. There were no between group differences in serious adverse events or adverse events leading to drug discontinuation. Investigator-reported prespecified adverse events included more frequent hepatic enzyme elevations (4.5% vs 2.6%) and renal adverse events (10.3% vs. 8.1%) in the bempedoic acid group. Myalgias were reported in 4.2% of bempedoic acid-treated patients compared with 5.9% of placebo-treated patients. A higher incidence of hyperuricemia (12.1% vs. 6.3%), gout (2.6% vs 2.0%) and cholelithiasis (2.5% vs 1.1%) occurred in the bempedoic acid group. Rates of new onset diabetes and the change in glycated hemoglobin levels in patients with diabetes at baseline were similar in both treatment groups.

TABLE 9
Investigator-Reported Adverse Events and Safety
Laboratory Findings (Safety Population)

| | | Placebo |
|---|---|---|
| | Bempedoic Acid | (N = 2101), |
| | (N = 2104), n (%) | n (%) |
| Serious treatment emergent | 418 (19.9) | 438 (20.8) |
| adverse event | | |
| Adverse event leading to | 209 (9.9) | 209 (9.9) |
| drug discontinuation | | |
| Any treatment emergent | 1785 (84.8) | 1744 (83.0) |
| adverse event | | |
| Worsening hyperglycemia^{a} | 297/1372 | 294/1408 |
| | (21.6) | (20.9) |
| Muscular disorders | 269 (12.8) | 291 (13.9) |
| Hyperuricemia | 254 (12.1) | 133 (6.3) |
| Renal impairment | 216 (10.3) | 170 (8.1) |
| New onset diabetes in | 45/538 | 46/548 |
| patients with pre- | (8.4) | (8.4) |
| diabetes at baseline^{b} | | |
| New onset diabetes in | 47/732 | 48/693 |
| patients without diabetes | (6.4) | (6.9) |
| at baseline | | |
| Myalgias | 88 (4.2) | 124 (5.9) |
| Hypoglycemia | 104 (4.9) | 81 (3.9) |
| Elevated hepatic enzymes | 94 (4.5) | 55 (2.6) |
| Malignancies | 84 (4.0) | 86 (4.1) |
| Atrial fibrillation | 53 (2.5) | 52 (2.5) |
| Gout | 55(2.6) | 41 (2.0) |
| Cholelithiasis | 53 (2.5) | 24 (1.1) |
| Tendinopathies | 37 (1.8) | 34 (1.6) |
| Discontinuation of | 29 (1.4) | 35 (1.7) |
| treatment due to myalgia | | |
| Adjudicated tendon rupture | 29 (1.4) | 18 (0.9) |
| Neurocognitive disorders | 9 (0.4) | 19 (0.9) |

Laboratory Results after 6 months, mg/dL

| | | |
|---|---|---|
| Change from baseline in | 0.80 (1.1) | −0.01 (1.0) |
| uric acid level, mean (SD) | | |
| Patients with uric acid | 215/1996 | 82/ |
| level >8.5 mg/dL, n (%) | (10.8%) | 1993(4.1%) |
| Change from baseline in | 0.05 (0.19) | 0.02 (0.14) |
| creatinine levels, mean (SD) | | |
| Patients with | 65/1996 | 52/1993 |
| creatinine >1.5 mg/dL, n (%) | (3.3%) | (2.6%) |

Laboratory Results after 12 months

| | | |
|---|---|---|
| Change from baseline in | 0.03 (0.79) | 0.06 (0.77) |
| glycated hemoglobin | | |
| level (%)^{c} | | |
| Change in patients | 0.02(0.26) | 0.06 (0.31) |
| without diabetes at baseline | | |
| Change in patients with | 0.03(0.96) | 0.06 (0.91) |
| diabetes at baseline | | |

Enzyme abnormalities at any visit, n (%)

| | | |
|---|---|---|
| Creatine kinase levels >10x | 5 (0.2) | 2 (0.1) |
| ULN (single occurrence) | | |
| Creatine kinase levels >10x | 0 | 0 |
| ULN (repeated and confirmed) | | |
| Alanine aminotransferase | 44 (2.1) | 40 (1.9) |
| level >3 times ULN^{d} | | |
| Aspartate aminotransferase | 72 (3.5) | 27 (1.3) |
| level >3 times ULN^{&} | | |

Abbreviations: upper limit of normal, ULN;
standard deviation, SD
^{a}In patients with diabetes at baseline
^{b}Patients with prediabetes at baseline were defined as: No past medical history of diabetes and with glycated hemoglobin level of ≥5.7% and <6.5% or 1 or more measurement of fasting glucose ≥100 mg/dL (5.6 mmol/L), but not more than 1 value of fasting glucose ≥126 mg/dL (7.0 mmol/L). Patients with normoglycemia at baseline did not meet criteria for prediabetes.
^{c}Not a prespecified safety measure
^{d}Repeated and confirmed
All reported adverse events other than the laboratory findings represent the investigator's judgement.
Specific guidance was not provided.

### Discussion

In patients with elevated cardiovascular risk, but without a prior event, this prespecified subgroup analysis showed that administration of bempedoic acid in patients who were unable or unwilling to take guideline recommended doses of a statin, was associated with a significant reduction in the primary end point, 4-component MACE (2.3% absolute risk reduction), NNT to prevent 1 primary event was 43 patients. This primary prevention cohort was a prespecified subgroup that represented 30% of the total enrolled within a larger trial that included a mixed population of primary and secondary prevention patients. Treatment was also associated with significant benefits for several key secondary end points, including the prespecified 3-component MACE (2.4% absolute risk reduction), and significant reductions in MI, cardiovascular death, and all-cause mortality. Stroke and coronary revascularization were not significantly reduced. Adverse events included a higher incidence of gout, hepatic enzyme elevations, and increased serum creatinine levels.

After 6 months of treatment, bempedoic acid, compared with placebo, reduced LDL cholesterol by 30.2 mg/dL (21.3%) and hsCRP by 0.56 mg/L (21.5%). Although the time-averaged difference in LDL cholesterol over the duration of the trial was moderate, 23.2 mg/dL (16.3%), there were substantial reductions in major adverse cardiovascular outcomes and mortality. These findings emphasize the potential value of lipid modulating therapy in patients with no prior cardiovascular event, but who have a high risk for a first event. In this study, hsCRP was significantly reduced.

In conclusion, in primary prevention patients unable to tolerate recommended doses of statins, bempedoic acid was associated with a significant reduction in the primary composite end point, time to death from cardiovascular causes, nonfatal MI, nonfatal stroke, or coronary revascularization. Treatment was also associated with significant reductions in MI, cardiovascular death, and all-cause mortality.

### Example 3—A Study Assessing the Cardiovascular Benefits and Risk of New Onset Diabetes by Glycemic Status in Patients Administered Bempedoic Acid

### 1) Methods

CLEAR OUTCOMES (described in Example 1) was a randomized double-blind trial comparing orally administered bempedoic acid 180 mg once daily to placebo in statin-intolerant patients with or at high risk of CVD and an LDL-C ≥2.59 mmol/L. This prespecified analysis reports the time to event analysis of bempedoic acid versus placebo on the primary endpoint (MACE-4; composite of CV death, nonfatal MI, nonfatal stroke, or coronary revascularization) and key secondary end-point (MACE-3; death from cardiovascular causes, nonfatal myocardial infarction, nonfatal stroke) by baseline glycaemia status (diabetes, prediabetes, and normoglycaemia), and impact on HbA1c at 1 and 3 years and on NOD for subjects without diabetes at baseline.

### 2) Results

A total of 13,970 patients were included in CLEAR Outcomes; 45.6% with diabetes, 41.5% with pre-diabetes, and 12.9% with normoglycaemia at baseline. Compared to placebo, LDL-C was reduced by 21.2%, 20.7% and 22.0% respectively at month 6 and in absolute terms by 0.73, 0.76, and 0.81 mmol/L, respectively (all p<0.0001). Table 10 reports the incidence of MACE-4 and MACE-3 endpoints by assigned treatment and baseline glycaemic and CVD risk strata over a median 3.4 year follow up. Overall bempedoic acid reduced MACE-4 and MACE-3 without evidence of effect modification across glycaemic strata, with greater absolute benefits among those with diabetes. The rate of NOD in patients without diabetes at baseline randomized to bempedoic acid was 11.1% vs 11.5% with placebo (HR 0.95; 95% CI 0.83,1.09). There was no impact on post-randomization fasting serum glucose or HbA1c levels at 12 or 36 months with bempedoic acid in patients considered to have pre-diabetes or normoglycaemia.

TABLE 10
MACE Endpoints by Baseline Glycaemic Status
and CVD risk group among diabetics

| Baseline | | | | |
|---|---|---|---|---|
| glycaemic | Bempedoic Acid | Placebo | | |
| status | N = 6992 | N = 6978 | HR | p |
| | Time to First Adjudicated MACE-4 | | | 0.42 |
| Normo- | 95/937 (10.1%) | 103/864 (11.9%) | 0.84 | 0.21 |
| glycemia | | | (0.63, 1.10) | |
| Pre-Diabetes | 350/2911 (12.0%) | 364/2885 (12.6%) | 0.94 | 0.42 |
| | | | (0.81, 1.09) | |
| Diabetes | 374/3144 (11.9%) | 460/3229 (14.3%) | 0.83 | 0.006 |
| | | | (0.72, 0.95) | |
| | Time to First Adjudicated MACE-3 | | | 0.41 |
| Normo- | 67/937 (7.2%) | 64/864 (7.4%) | 0.96 | 0.81 |
| glycemia | | | (0.68, 1.35) | |
| Pre-Diabetes | 229/2911 (7.9%) | 244/2885 (8.5%) | 0.92 | 0.35 |
| | | | (0.77, 1.10) | |
| Diabetes | 279/3144 (8.9%) | 355/3229 (11.0%) | 0.80 | 0.005 |
| | | | (0.68, 0.93) | |
| CVD risk | | | | |
| group | Bempedoic Acid | Placebo | | |
| (Diabetes) | N = 3144 | N = 3229 | HR | p |
| | Time to First Adjudicated MACE-4 | | | 0.18 |
| Primary | 87/1369 (6.4%) | 127/1412 (9.0%) | 0.70 | 0.01 |
| Prevention | | | (0.53, 0.92) | |
| Secondary | 287/1775 (16.2%) | 333/1817 (18.3%) | 0.87 | 0.09 |
| Prevention | | | (0.74, 1.02) | |
| | Time to First Adjudicated MACE-3 | | | 0.12 |
| Primary | 67/1369 (4.9%) | 106/1412 (7.5%) | 0.65 | 0.004 |
| Prevention | | | (0.48, 0.88) | |
| Secondary | 212/1775 (11.9%) | 249/1817 (13.7%) | 0.86 | 0.11 |
| Prevention | | | (0.72, 1.03) | |

### 3) Conclusions

Patients with statin intolerance and diabetes derive greater absolute benefit from bempedoic acid. There was no increase in NOD and no worsening of HbA1c with bempedoic acid in those without DM. Among high-risk patients, such as those with diabetes and unable or unwilling to take statins, bempedoic acid is a safe, effective therapeutic oral option to reduce the risk of cardiovascular disease.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

We claim:

1. A method of reducing the risk of non-fatal myocardial infarction in an adult with established cardiovascular disease or without established cardiovascular disease and at high risk for a cardiovascular disease event, the method comprising orally administering daily to the adult a pharmaceutical formulation comprising 180 mg bempedoic acid and one or more pharmaceutically acceptable excipients, wherein administration of the pharmaceutical formulation reduces the risk of non-fatal myocardial infarction by at least about 25% as compared to a placebo in a human population with established cardiovascular disease or without established cardiovascular disease and at high risk for a cardiovascular disease event.

2. The method of claim 1, wherein the adult has established cardiovascular disease.

3. The method of claim 1, wherein the adult is without established cardiovascular disease and at high risk for a cardiovascular disease event.

4. The method of claim 3, wherein the adult has diabetes mellitus.

5. The method of claim 3, wherein the adult has a Reynolds Risk score of greater than about 30%.

6. The method of claim 3, wherein the adult has a SCORE risk score of greater than about 7.5%.

7. The method of claim 3, wherein the adult has two or more risk factors for a cardiovascular disease event.

8. The method of claim 7, wherein the two or more risk factors for a cardiovascular disease event are selected from the group consisting of gender, age, smoking status, systolic blood pressure, total cholesterol, high-density lipoprotein (HDL) cholesterol, high sensitivity C-reactive protein (hsCRP), low-density lipoprotein cholesterol (LDL-C), and familial history of cardiovascular disease events.

9. The method of claim 1, wherein the adult has primary hyperlipidemia.

10. The method of claim 9, wherein the primary hyperlipidemia is heterozygous familial hypercholesterolemia.

11. The method of claim 1, further comprising administering to the adult another low-density lipoprotein cholesterol (LDL-C) lowering therapy.

12. The method of claim 11, wherein the other LDL-C-lowering therapy comprises a statin.

13. The method of claim 11, wherein the other LDL-C-lowering therapy comprises ezetimibe.

14. The method of claim 1, wherein the pharmaceutical formulation further comprises another low-density lipoprotein cholesterol (LDL-C) lowering therapy.

15. The method of claim 14, wherein the other LDL-C-lowering therapy comprises ezetimibe.

16. The method of claim 1, wherein the adult is statin intolerant.

17. The method of claim 1, wherein the pharmaceutical formulation is administered to the adult once daily.

18. The method of claim 1, wherein the administration of the pharmaceutical formulation reduces the risk of non-fatal myocardial infarction by about 27% as compared to a placebo in a human population with established cardiovascular disease or without established cardiovascular disease and at high risk for a cardiovascular disease event.
